(19) United States
(12) Patent Application Publication
(10) Pub. No.: US 2013/0216482 A1
(43) Pub. Date: Aug. 22, 2013

(54) METHODS AND COMPOSITIONS FOR NON-INVASIVE, DYNAMIC IMAGING OF INTESTINAL MOTILITY

(71) Applicant: The Board of Regents of the University of Texas System, (US)

(72) Inventors: Sunkuk KWON, Houston, TX (US); Eva M. SEVICK-MURACA, Houston, TX (US); Cynthia DAVIES-VENN, Houston, TX (US)

(73) Assignee: The Board of Regents of the University of Texas System, Austin, TX (US)

(21) Appl. No.: 13/758,637

(22) Filed: Feb. 4, 2013

Related U.S. Application Data
(60) Provisional application No. 61/594,880, filed on Feb. 3, 2012.
(60) Provisional application No. 61/650,805, filed on May 23, 2012.

(51) Int. Cl. A61B 5/00

(52) U.S. Cl. CPC A61B 5/0071; A61B 5/4255
USPC 424/9.6

(57) ABSTRACT

Described are methods and composition for use in non-invasive imaging of intestinal structure and function. These methods can be used to identify, diagnose, assess, monitor and direct therapies for gastrointestinal diseases and disorders. Embodiments of the methods utilize highly sensitive optical imaging and fluorescent spectroscopy techniques to track or monitor packets of organic dye excreted in bile into the intestinal tract to provide quantitative information regarding intestinal propulsion and function.

This application claims the benefit of U.S. Provisional Patent Application Nos. 61/594,880, filed Feb. 3, 2012, and 61/650,805, filed May 23, 2012, the entirety of which are incorporated herein by reference.

The invention was made with U.S. government support under Grant Nos. DK056338, HL092923, CA128919, and CA136404 awarded by the National Institutes of Health. The government has certain rights in the invention.

### BACKGROUND OF THE INVENTION

1. Field of the Invention

The present invention relates generally to the field of biomedical imaging. More particularly, it concerns methods and compositions for functionally imaging and measuring intestinal motility in an individual using a highly sensitive near infra-red (NIR) imaging system. This disclosure also relates to the use of such methods and compositions in the diagnosis, assessment, and monitoring of diseases and disorders of the gastrointestinal tract using administration of a near infra-red (NIR) fluorescent dye and a highly sensitive imaging system.

2. Description of Related Art

The gastrointestinal (GI) tract has various controlled motions, such as peristaltic and segmental motions, to mix and propel its contents as an essential part of digestion and absorption. Different patterns of intestinal motions, such as peristaltic and segmental, have been described and are shown to be the result of interplay among smooth muscle cells, interstitial cells of Cajal (ICCs), and enteric neural circuits (Huizing a, J. D., Thuneberg, L., Vanderwinden, J. M. and Rumessen, J. J., “Interstitial cells of Cajal as targets for pharmacological intervention in gastrointestinal motor disorders,” Trends Pharmacol Sci. 18, 393-403, 1997). The peristaltic motions represent aborally propagating contraction waves that push contents forward. In contrast, segmental motions are stationary and represent constrictions at fixed spatial locations along the GI tract, allowing greater mixing of the food chyme. Phasic contractions are caused by electrical activity, termed “slow waves,” which originate in the ICCs that form a network of cells distributed in the GI tract (Huizing a, J. D., Thuneberg, L., Kluppel, M., Malysz, J., Mikkelsen, H. B. and Bernstein, A., “W/kit gene required for interstitial cells of Cajal and for intestinal pacemaker activity,” Nature 373, 347-349, 1995). Normal intestinal motility can be influenced by pathological conditions including irritable bowel syndrome (IBS), inflammatory bowel disease (IBD), and chronic intestinal pseudo-obstruction (Bratten, J. R. and Jones, M. P., “Small intestinal motility,” Curr Opin Gastroenterol 23, 127-133, 2007; Husebye, E., “The patterns of small bowel motility: physiology and implications in organic disease and functional disorders,” Neurogastroenterol Motil 11, 141-161, 1999), resulting in changes of slow wave activity. Thus, it has been proposed that ICCs could be ideal targets for pharmacological intervention in GI motility dysfunction.

Intestinal motility function has been extensively assessed on the isolated segments of small laboratory animal intestines by measuring electrical signals from the GI muscles with or without video imaging for construction of spatio-temporal maps of changes in diameter (Seerden, T. C., et al., Am J Physiol Gastrointest Liver Physiol 289, G1043-1051, 2005; Hennig, G. W., et al., J Physiol 517 (Pt 2), 575-590, 1999; Huizing a, J. D., et al., J Physiol 506 (Pt 3), 843-856, 1998; Grivel and Ruckebusch, J Physiol 227, 611-625, 1972). However, these ex vivo and in situ measurements while informative are invasive and do not enable longitudinal monitoring of GI motility in disease and response to pharmacological intervention but certainly have no utility in the clinic.

Several groups have reported non-invasive dynamic x-ray imaging of intestinal motility in conscious mice after oral gavage of barium or iodine solution (Der-Silaphet, T., et al., Gastroenterology 114, 724-736, 1998; Der, T., et al., Gastroenterology 119, 1590-1599, 2000; Bercik, P., et al., Gastroenterology 127, 179-187, 2004). Slow wave driven peristaltic activities in the small intestine were radiographically observed after the stomach emptied of contrast fluid. However there were difficulties associated with repeated oral contrast agent administration and art factual intestinal motions were observed due to the distension of the intestinal walls by the significant quantities of contrast required.

Thus while some experimental laboratory animal models have been developed to study gut motility and how it may be impacted by potential pharmacological agents, there remains a need for non-invasive and long-term assessment tools of intestinal motility. Particularly needed are techniques that can be used in the clinic on a surviving human or veterinary patient.

Current practices to define the anatomy of the intestinal tract use X-ray, CT or MRI in order to rule out structural problems and then evaluate GI tract functions using different techniques for different types of information, such as fluoroscopy, endoscopy, scintigraphy, breath hydrogen measurement, manometry, and electrogastrography. However, several of these require the contrast agents many of which are radioactive. For example, scintigraphic evaluation of gastric emptying with oral administration of Tc99m labeled compound is widely used in the human clinical practice but it is less useful in veterinary clinical practice due mainly to long camera integration times and low temporal resolution.

Therefore there is an unmet need for non-invasive imaging of intestinal contractile function with sufficient sensitivity to identify abnormal patterns and physiology, provide correct diagnosis of GI motility disorders or dysfunction, and to guide evaluation of pharmacologic and surgical therapy in both veterinary and human patients.

### SUMMARY OF THE INVENTION

The problem of non-invasively imaging gastrointestinal (GI) motility without the use of radioactive contrast agents, but with sufficient temporal resolution and sensitivity to assess dynamic motion of the intestine can be solved using administration of a highly sensitive NIR fluorescent dye and imaging system in combination with auto fluorescent compounds present in foodstuffs. Such compositions can be used to identify abnormal patterns and physiology, provide correct diagnosis of GI motility disorders or dysfunction, and guide evaluation of pharmacologic and surgical therapy for GI motility diseases, disorders, and related symptoms in both veterinary and human patients.

One embodiment of the present invention provides a method of non-invasively imaging gastrointestinal motility in an animal having an abdominal surface and a gastrointestinal tract. For example, a method can comprise the steps of: (a) administering an animal foodstuff containing at least one auto fluorescent compound to the animal, (b) allowing sufficient time for said foodstuff to enter into the gastrointestinal tract, (c) illuminating the abdominal surface with an excitation light to excite the auto fluorescent compound, (d) continuously and non-invasively detecting fluorescent emissions from the auto fluorescent compound to image at least a region of the gastrointestinal tract, (e) capturing a plurality of images of said one or more regions of the gastrointestinal tract based on said detected auto fluorescent images, and (f) tracking in vivo propulsion of said auto fluorescent compound through the one or more regions of the gastrointestinal tract based on said captured images.

In a further embodiment, the present invention provides a method of non-invasively imaging gastrointestinal motility in an animal, the method comprising the steps of: (a) administering to said animal at least one packet containing a fluorescent agent that when administered in vivo is excreted in bile; (b) allowing sufficient time for said at least one packet containing a fluorescent agent to be processed by the liver and excreted in bile; (c) illuminating the abdominal tissue surface with an excitation light to excite the fluorescent agent; (d) continuously and non-invasively detecting fluorescent emissions from the fluorescent agent to image at least one region of the intestinal tract; (e) capturing a plurality of images of said one or more regions of the intestinal tract based on said detected fluorescent images; and (1) tracking in vivo propulsion of said at least one packet through the one or more regions of the intestinal tract based on said captured images.

In certain aspects, an auto fluorescent compound can have an excitation wavelength ranging from about 700 nm to about 900 nm. Continuously and non-invasively detecting may comprise, for example, using an intensified charge-coupled camera.

In certain aspects, the excitation light source used to illuminate the abdominal surface may be laser diodes, semiconductor laser diodes, gas lasers, light emitting diodes, or combinations thereof.

In some aspects, the capturing a plurality of images comprises a camera integration time in the range of about 10 milliseconds to about 1 second. Preferably, the capturing a plurality of images comprises a camera integration time in the range of about 100 milliseconds to about 800 milliseconds. More preferably, the capturing a plurality of images comprises a camera integration time greater than 200 milliseconds.

In further aspects, the method further comprises assessing intestinal functionality by calculating an intestinal flow velocity. In another aspect, the method further comprises assessing intestinal functionality by calculating an intestinal peristaltic wave rate. In yet another aspect, the method further comprises assessing intestinal functionality by calculating an intestinal segmental contraction rate.

In another embodiment, the present invention provides a method of non-invasively assessing gastrointestinal function in an individual, the method comprising the steps of: (a) administering feedstuff containing at least one auto fluorescent compound to the individual, the at least one auto fluorescent compound having a characteristic excitation wavelength and a characteristic fluorescence emission wavelength; (b) non-invasively illuminating a tissue surface of the intestinal tract on the individual's body with radiation at said characteristic excitation wavelength; (c) non-invasively detecting fluorescence emissions from said compounds within a region of interest and capturing a plurality of fluorescence images for an interval of time; (d) using said fluorescence images to visualize gastrointestinal motion in a region of interest and to track the location of said compounds in said region of interest as a function of time to obtain a set of tracked image locations as a function of time; (e) determining from said tracked locations as a function of time an initial intestinal propulsion measurement; (f) optionally, comparing said initial intestinal propulsion measurement to a subsequently determined intestinal propulsion measurement; and (g) determining from the results of (d) and (e), and (f) if present, the functionality of a gastrointestinal tract in said region of interest in said individual.

In one aspect, the using said fluorescence images to visualize gastrointestinal motion in a region of interest comprises capturing images at an integration time ranging from about 10 ms to about 1 s.

In one aspect, the characteristic excitation wavelength of the auto fluorescent compound is in the region of 750-800 nm, and the characteristic fluorescence emission wavelength of the auto fluorescent compound is greater than 800 nm.

In certain aspects, the intestinal propulsion measurements may be intestinal peristaltic wave rate or segmental contraction rate.

In one case, the initial intestinal propulsion measurement comprises an initial peristaltic wave rate, which is compared to a subsequently determined intestinal peristaltic wave rate to determine the functionality of a gastrointestinal structure, wherein the intestinal function in said region of interest is improved if the subsequent intestinal peristaltic wave rate is greater than the initial intestinal peristaltic wave rate. In another case, the initial intestinal propulsion measurement comprises an initial intestinal segmental contraction rate, which is compared to a subsequently determined intestinal segmental contraction rate to determine the functionality of a gastrointestinal structure, wherein the intestinal function in said region of interest is improved if said subsequent intestinal segmental contraction rate is greater than said initial intestinal segmental contraction rate. In one aspect, the individual is administered a treatment to ameliorate the symptoms of a gastrointestinal disease or disorder after obtaining the initial measurement but before obtaining the second measurement. In another aspect, an intestinal propulsion measurement of less than a control value indicates the presence of a gastrointestinal disease or disorder.

In one embodiment, the methods may be used to non-invasively assess gastrointestinal motility in an individual. In another embodiment, the methods may be used to non-invasively measure propulsion of the gastrointestinal system of a patient comprises at least one of contraction frequency and the propagation velocity. In yet another embodiment, the methods may be used to non-invasively identify a patient suffering from a gastrointestinal motility disorder. In yet another embodiment, the methods may be used to non-invasively identify a patient at risk of a gastrointestinal motility disorder. In yet another embodiment, the methods may be used to non-invasively identify a patient in need of therapy for a gastrointestinal motility disorder. In yet another embodiment, the methods may be used to non-invasively identify a patient in need of surgery for a gastrointestinal blockage. In yet another embodiment, the methods may be used to non-invasively identify the portion of a patient's intestinal tract at risk of developing a gastrointestinal motility disorder. In yet another embodiment, the methods may be used to non-invasively identify the particular portion of a patient's intestinal tract in need of therapy due to a gastrointestinal motility disorder. In yet another embodiment, the methods may be used to non-invasively identify the particular portion of a patient's intestinal tract in need of surgery due to a gastrointestinal motility disorder. In yet another embodiment, the methods may be used to non-invasively determine the efficacy of a therapy for a gastrointestinal motility disorder in a patient being treated for a gastrointestinal motility disorder. In yet another embodiment, the methods may be used to non-invasively monitor the progress of a patient being treated for a gastrointestinal motility.

In one embodiment, the methods may be used to accelerate both preclinical and clinical drug or therapy discovery, validation, and approval by providing a convenient method for longitudinally assessing gastrointestinal motility in veterinary or human patients.

In one aspect, the individual is an animal. Preferably, the animal is a mammal. More preferably, the mammal is a human.

In one aspect, the fluorescent agent may comprise indocyanine green “ICG”). In another aspect, the fluorescent agent may have an excitation wavelength ranging from about 700 nm to about 900 nm.

In one aspect, the fluorescent agent can be injected intradermally. In another aspect, the fluorescent agent can be administered intravenously.

In one aspect, continuously and non-invasively detecting may comprise, for example, using an intensified charge-coupled camera.

In certain aspects, the excitation light source used to illuminate the abdominal surface may be laser diodes, semiconductor laser diodes, gas lasers, light emitting diodes, or combinations thereof.

In one aspect, the capturing a plurality of images comprises a camera integration time in the range of about 10 milliseconds to about 1 second. Preferably, the capturing a plurality of images comprises a camera integration time in the range of about 100 milliseconds to about 800 milliseconds. More preferably, the capturing a plurality of images comprises a camera integration time greater than 200 milliseconds.

In one aspect, the method further comprises assessing intestinal functionality by calculating an intestinal flow velocity. In another aspect, the method further comprises assessing intestinal functionality by calculating an intestinal peristaltic wave rate. In yet another aspect, the method further comprises assessing intestinal functionality by calculating an intestinal segmental contraction rate.

In another embodiment, the invention provides a method of non-invasively assessing gastrointestinal function in an individual, the method comprising the steps of: (a) administering to said animal at least one packet containing a fluorescent agent having a characteristic excitation wavelength and a characteristic fluorescence emission wavelength and that when administered in vivo is excreted in bile; (b) allowing sufficient time for said at least one packet containing the fluorescent agent to be processed and excreted in bile; (c) noninvasively illuminating a tissue surface of the intestinal tract on the individual's body with radiation at said characteristic excitation wavelength; (d) noninvasively detecting fluorescence emissions from said agent within a region of interest and capturing a plurality of fluorescence images for an interval of time; (e) using said fluorescence images to visualize gastrointestinal motion in a region of interest and to track the location of said agent in said region of interest as a function of time to obtain a set of tracked image locations as a function of time; (f) determining from said tracked locations as a function of time an initial intestinal propulsion measurement; (g) optionally, comparing said initial intestinal propulsion measurement to a subsequently determined intestinal propulsion measurement; and (h) determining from the results of (e) and (f), and (g) if present, the functionality of a gastrointestinal tract in said region of interest in said individual.

In one aspect, the fluorescent agent may comprise indocyanine green (“ICG”). In another aspect, the fluorescent agent may have a characteristic excitation wavelength ranging from about 750 nm to about 800 nm and a characteristic fluorescence emission wavelength greater than 800 nm.

In one aspect, the capturing a plurality of images comprises a camera integration time in the range of about 10 milliseconds to about 1 second.

In certain aspects, the intestinal propulsion measurements may be intestinal peristaltic wave rate or segmental contraction rate.

In one case, the initial intestinal propulsion measurement comprises an initial peristaltic wave rate, which is compared to a subsequently determined intestinal peristaltic wave rate to determine the functionality of a gastrointestinal structure, wherein the intestinal function in said region of interest is improved if the subsequent intestinal peristaltic wave rate is greater than the initial intestinal peristaltic wave rate. In another case, the initial intestinal propulsion measurement comprises an initial intestinal segmental contraction rate, which is compared to a subsequently determined intestinal segmental contraction rate to determine the functionality of a gastrointestinal structure, wherein the intestinal function in said region of interest is improved if said subsequent intestinal segmental contraction rate is greater than said initial intestinal segmental contraction rate. In one aspect, the individual is administered a treatment to ameliorate the symptoms of a gastrointestinal disease or disorder after obtaining the initial measurement but before obtaining the second measurement. In another aspect, an intestinal propulsion measurement of less than a control value indicates the presence of a gastrointestinal disease or disorder.

In one embodiment, the methods may be used to non-invasively assess gastrointestinal motility in an individual. In another embodiment, the methods may be used to non-invasively measure propulsion of the gastrointestinal system of a patient comprises at least one of contraction frequency and the propagation velocity. In yet another embodiment, the methods may be used to non-invasively identify a patient suffering from a gastrointestinal motility disorder. In yet another embodiment, the methods may be used to non-invasively identify a patient at risk of a gastrointestinal motility disorder. In yet another embodiment, the methods may be used to non-invasively identify a patient in need of therapy for a gastrointestinal motility disorder. In yet another embodiment, the methods may be used to non-invasively identify a patient in need of surgery for a gastrointestinal blockage. In yet another embodiment, the methods may be used to non-invasively identify the portion of a patient's intestinal tract at risk of developing a gastrointestinal motility disorder. In yet another embodiment, the methods may be used to non-invasively identify the particular portion of a patient's intestinal tract in need of therapy due to a gastrointestinal motility disorder. In yet another embodiment, the methods may be used to non-invasively identify the particular portion of a patient's intestinal tract in need of surgery due to a gastrointestinal motility disorder. In yet another embodiment, the methods may be used to non-invasively determine the efficacy of a therapy for a gastrointestinal motility disorder in a patient being treated for a gastrointestinal motility disorder. In yet another embodiment, the methods may be used to non-invasively monitor the progress of a patient being treated for a gastrointestinal motility.

In one embodiment, the methods may be used to accelerate both preclinical and clinical drug or therapy discovery, validation, and approval by providing a convenient method for longitudinally assessing gastrointestinal motility in veterinary or human patients.

In one aspect, the individual is an animal. Preferably, the animal is a mammal. More preferably, the mammal is a human.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1A: Overlay of white light and fluorescent images generated using in vivo NIR imaging of auto fluorescent compounds located within foodstuff in a mouse. The location of the Cecum is identified by the asterisk (*).

FIG. 1B: Overlay of white light and fluorescent images generated using ex vivo NIR imaging of auto fluorescent compounds within the foodstuff located within the excised GI tract of a mouse. The location of the Cecum is identified by the asterisk (*).

FIG. 1C: Magnified fluorescent image of the area indicated by the white rectangle in FIG. 1B, illustrating NIR imaging of auto fluorescence produced by diet containing chlorophyll. Inset demonstrates an overlay of white light and fluorescent images of chlorophyll free diet (on left) and chlorophyll containing standard diet (on right). Scale bar=1 mm.

FIG. 2A: Fluorescent images of peristaltic motion in a ventral view of the GI tract of a mouse generated using NIR imaging of auto fluorescent compounds in the foodstuff.

FIG. 2B: Fluorescent images of peristaltic motion in the GI tract of a mouse generated using highly sensitive NIR imaging of auto fluorescent compounds in the food stuff and after selection of 46 ROIs along the fluorescent small intestine. Arrow indicates the location where peristaltic motion occurred.

FIG. 2C: A 3-D plot of fluorescent intensity as a function of time and ROI showing the propagation of the contractile waves as indicated by a red arrow.

FIG. 2D: Fluorescent images of segmental motion in a ventral view of the GI tract of a mouse generated using highly sensitive NIR imaging of auto fluorescent compounds in the foodstuff.

FIG. 2E: Fluorescent images of segmental motion in the GI tract of a mouse generated using highly sensitive NIR imaging of auto fluorescent compounds in the foodstuff and after selection of 46 ROIs along the fluorescent small intestine. Arrow indicates the location where segmental motion occurred.

FIG. 2F: A 3-D plot of fluorescent intensity as a function of time and ROI demonstrating segmental motion as indicated by arrows.

FIG. 3A: Fluorescent image frame showing blood vessels 4 secs after intravenous injection of ICG. Arrow: superficial blood vessels. Broken arrow: blood vessels in the GI.

FIG. 3B: Fluorescent intensity plot as a function of time, showing differential clearance rate of ICG following intravenous (top red line) and intradermal injection (bottom blue line).

FIG. 4A: Overlay of white light and fluorescent images showing the biliary system and the intestine in a mouse with exposure of the abdomen 1 hr after intravenous injection.

FIG. 4B: Fluorescent images showing the biliary system and the intestine in a mouse with exposure of the abdomen 1 hr after intravenous injection.

FIG. 4C: Magnified fluorescent image of the rectangle in FIG. 1B showing the biliary system. Scale bar=1 mm. CBD: common bile duct. CD: cystic duct. DU: duodenum. GA: gallbladder. HD: hepatic duct.

FIG. 4D: Overlay of white light and fluorescent images of isolated GI tract from the body. The small intestine was cut into three segments and contents were removed. CBD: common bile duct. CE: cecum. GA: gallbladder. HD: hepatic duct. IT: intestinal tissues. LI: liver.

FIG. 5A: Overlay of white light and fluorescent images 30 min, 1 h, 3 h, 5 h, and 24 h after intravenous and intradermal injection of ICG, showing GI transit of ICG.

FIG. 5B: Fluorescent image showing the placement of 76 ROIs on the fluorescent small intestine.

FIG. 5C: Fluorescent intensity map as a function of time and the length of the intestine, demonstrating aborally propagating peristaltic waves (solid arrows).

FIG. 5D: Fluorescent intensity map as a function of time and the length of the intestine, demonstrating segmental contractions (solid arrows).

FIG. 6: Schematic of custom-built NIR fluorescence imaging system.

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### DESCRIPTION OF ILLUSTRATIVE EMBODIMENTS

As used herein, and unless otherwise indicated, the terms “treat”, “treating”, “treatment” and “therapy” contemplate an action that occurs while a patient is suffering from a gastrointestinal motility diseases or disorder s that reduces the severity of one or more symptoms or effects of a gastrointestinal motility disease or disorder, or a related disease or dysfunction. Where the context allows, the terms “treat”, “treating”, and “treatment” also refers to actions taken toward ensuring that individuals at increased risk of a gastrointestinal motility disease or disorder are able to receive appropriate surgical and/or other medical intervention prior to onset of a gastrointestinal motility disease or disorder. As used herein, and unless otherwise indicated, the terms “prevent”, “preventing”, and “prevention” contemplate an action that occurs before a patient begins to suffer from a gastrointestinal motility disease or disorder that delays the onset of, and/or inhibits or reduces the severity of, a gastrointestinal motility disease or disorder. As used herein, and unless otherwise indicated, the terms “manage”, “managing”, and “management” encompass preventing, delaying, or reducing the severity of a recurrence of a gastrointestinal motility disease or disorder in a patient who has already suffered from such a disease or condition. The terms encompass modulating the threshold, development, and/or duration of a gastrointestinal motility disease or disorder or changing how a patient responds to a gastrointestinal motility disease or disorder.

As used herein, and unless otherwise specified, a “therapeutically effective amount” of a compound is an amount sufficient to provide any therapeutic benefit in the treatment or management of a gastrointestinal motility disease or disorder or to delay or minimize one or more symptoms associated with a gastrointestinal motility disease or disorder. A therapeutically effective amount of a compound means an amount of the compound, alone or in combination with one or more other therapies and/or therapeutic agents that provide any therapeutic benefit in the treatment or management of a gastrointestinal motility disease or disorder, or related diseases or dysfunction. The term “therapeutically effective amount” can encompass an amount that alleviates a gastrointestinal motility disease or disorder, improves or reduces lymphatic disorders, improves overall therapy, or enhances the therapeutic efficacy of another therapeutic agent.

As used herein, and unless otherwise specified, a “prophylactically effective amount” of a compound is an amount sufficient to prevent or delay the onset of a gastrointestinal motility disease or disorder, or one or more symptoms associated with a gastrointestinal motility disease or disorder or prevent or delay its recurrence. A prophylactically effective amount of a compound means an amount of the compound, alone or in combination with one or more other treatment and/or prophylactic agent that provides a prophylactic benefit in the prevention of a gastrointestinal motility disease or disorder. The term “prophylactically effective amount” can encompass an amount that prevents gastrointestinal motility disease or disorder, improves overall prophylaxis, or enhances the prophylactic efficacy of another prophylactic agent.

As used herein, and unless otherwise specified, the term “functional imaging” refers to how the structures function in terms of update of dye, the flow as determined by the dye, dynamics of flow, and direction of flow and the associated materials carried by it. The function of the gastrointestinal system can be described by velocity, period or frequency of propulsive events, peristaltic wave rate, segmental contraction rate, distention, contraction, permeability, and other parameters that provide evidence of dysfunction in comparison to normal function as imaged in healthy control animal or human subjects.

As used herein, a fluorescence imaging or collection device refers to a system which illuminates the surface of multiply scattering systems, such as biological tissues, with excitation light, and which collects the generated fluorescence from the surface, sub-surface, or deeper portions of the tissue.

As used herein, scattering is the general physical process where some forms of radiation, such as light, are forced to deviate from a straight trajectory by one or more localized non-uniformities in the medium through which they pass.

As used herein, “gastrointestinal motility disease or disorder” includes, but is not limited to, achalasia, aerophagia, anismus, Barrett's Syndrome, bile reflux, biliary dyskinesia, celiac disease, Crohn's disease, chronic abdominal pain, acute and chronic intestinal pseudo-obstruction (CIP), colitis, colonic inertia, constipation (acute and chronic), cyclic vomiting syndrome (CVS), diarrhea, diffuse esophageal spasm (DES), diverticulitis, dumping syndrome, dyspepsia, dysphagia, encopresis, enteric dysmotility, fecal incontinence, functional bowel outlet obstruction, functional fecal retention, gastric dysrelaxation, gastroesophageal reflux disease (GERD), gastroparesis, gastropathy, Hirschsprung's disease (HD), hypercontractile motility, hypermotility, hypertensive lower esophageal sphincter, hypomotility, ileus (functional or paralytic, post-operative), ineffective esophageal motility, intestinal dysmotility, intestinal ischemia, intestinal obstruction, intestinal pseudo-obstruction, irritable bowel syndrome (IBS), inflammatory bowel disease, megacolon, mitochondrial disease, neuronal intestinal dysplasia, non-erosive reflux disease, nutcracker esophagus, obstipation, Ogilvie syndrome, pancreatitis, pelvic floor dysfunction, short bowel syndrome, small bowel bacterial overgrowth, small bowel intestinal motility disorder, sphincter of Oddi dysfunction, slow transit constipation, superior mesenteric artery syndrome, and volvulus.

As used herein, “animal” refers to those animals that can suffer a gastrointestinal motility disease or disorder. Animals includes, but is not limited to, laboratory animals (such as, but not limited to, mice, rats, gerbils, hamsters, guinea pig, rabbits, etc.), livestock animals (such as, but not limited to, horses, cows, pigs, hogs, goat sheep, mules, llamas, etc), zoo or wild animals (such as, but not limited to, lizards and other reptiles, zebra, white-tailed deer, elk, moose, red deer, reindeer (caribou), fallow deer, roe deer, chital, sheep and goat, giraffe, elephant, wolf, fox, coyote, lion, tiger, leopard, cougar, bobcat, bear, monkey, chimpanzee, orangutans, gorillas, etc.), companion animals (such as, but not limited to, dogs, cats, ferrets, etc.) and humans.

The problem of non invasively imaging GI motility without the use of exogenous, radioactive contrast agents, but with sufficient temporal resolution and sensitivity to assess dynamic motion of the intestine is solved using administration of a NIR fluorescent dye and a highly sensitive NIR imaging system that is able to utilize the auto fluorescent properties of compounds within foodstuffs to detect intestinal motions and can be used to identify abnormal patterns and physiology, provide correct diagnosis of GI motility diseases, disorders, or dysfunction, and guide evaluation of pharmacologic and surgical therapy for both veterinary and human patients suffering or potentially suffering from a gastrointestinal motility disease or disorder.

Described are methods and compositions for use in non-invasive imaging of intestinal contractile function with the simple administration of a NIR fluorescent dye and using the auto fluorescent properties of compounds within foodstuffs in combination with a highly sensitive NIR imaging system that can be used to identify abnormal patterns and physiology, provide correct diagnosis of a gastrointestinal motility disease or disorder, and guide evaluation of pharmacologic and surgical therapy for both veterinary and human patients.

Recently the inventors developed a dynamic near-infrared (NIR) fluorescence imaging technique with injection of indocyanine green (ICG), which has been used to assess lymphatic function in both animals and humans (see for example, U.S. Pat. Nos. 5,865,754; 7,054,002; 7,328,059; US Patent Application Publication Nos: 2007/0286468; 2008/0056999; 2008/0064954; 2008/0175790 and 2011/007140). ICG is a NIR fluorophore that is excited at 780 nm and emits fluorescence at 830 nm, which is advantageous for non-invasive tissue imaging due to low tissue absorption and scattering and lack of background fluorescence.

ICG is a tricarbocyanine dye that can be excited between 760 and 785 nm to produce an emitted fluorescent signal that can be imaged between 820 and 840 nm. ICG is approved by the U.S. Food and Drug Administration (FDA) for hepatic and ophthalmologic diagnostic applications. ICG has been used safely for more than forty years. ICG's fluorescent properties and its dark green color also make it useful for vascular imaging/clearance studies following intravenous (i.v.) administration which results in its association with the plasma protein albumin. Thus, ICG is typically administered systemically in adults at total doses not exceeding 2 mg/kg. With a normal biological half-life of 2-4 min, it is then taken up by the liver and secreted into the bile and as bile enters the duodenum via the biliary tract in most animals, ICG-laden fluorescent bile can be used delineate the intestine, enabling non-invasive imaging of intestinal motility. Presently disclosed are methods and compositions that facilitate non-invasive, dynamic NIR fluorescence imaging of contractile activity of the intestinal tract following intravenous or intradermal injection of ICG (Kwon, S., et al., Neurogastroenterol Motil 2011, 23, 881-e344). These methods and compositions can be used to longitudinally and quantitatively access gastrointestinal motility in animals. In certain embodiments these methods and compositions may be radioactive contrast agent free.

Provided are compositions and methods that allow non-invasive NIR fluorescence imaging with sufficient temporal resolution and sensitivity to assess dynamic motion of the intestinal tract with or without contrast agents and, in certain instances, using only the auto fluorescence of certain compounds within foodstuffs. Delineation of the intestinal wall results from the secretion of bile containing ICG from the liver following intravenous or intradermal injection of ICG. In some embodiments these methods allow non-invasive NIR fluorescence imaging with sufficient temporal resolution and sensitivity to assess dynamic motion of the intestine as the result of delineation of the intestinal wall resulting from the secretion of bile containing ICG from the liver following intravenous or intradermal injection of ICG. In other embodiments, these methods and compositions use NIR imaging to allow the removal of the need for the injection of an exogenous imaging of any type, other than that which occur naturally in some foodstuffs.

The inventors have addressed the issue of limiting imaging sensitivity, non-invasively in certain instances, by improving imaging instrument design (Zhu B., et al., Med Phys 37, 5961-5970, 2010), to overcome the estimated at 4-5 centimeters of tissue depth from actual human studies (Sevick-Muraca, E. M., et al., Radiology 246, 734-741, 2008).

To validate the present method it was used to characterize the GI motility in the laboratory mouse fluorescence imaging of the intestinal tract. It was determined that contraction frequency and the propagation velocity are 35 cycles/min and 0.82±0.5 cm/s in the proximal small intestine and 27 cycles/min and 2.04±1.15 cm/s in the middle portion of the small intestine of the mouse. These values are in general agreement with previous findings of the propagation velocity and frequency of the wave in various mouse strains using in vivo dynamic fluoroscopy (Der-Silaphet, T., et al., Gastroenterology 114, 724-736, 1998; Der, T., et al., Gastroenterology 119, 1590-1599, 2000 and Bercik, P., et al., Gastroenterology 127, 179-187, 2004 and summarized in Table 1 below). In addition, data from ex vivo measurements following isolation of the small intestine showed the oscillation frequencies ranging from of 36.3±4.5 to 47.3±3.9 cycles/min and oscillation velocities of 1.00±0.37 to 1.64±0.42 cm/s, depending upon the location of the small intestine (Seerden, T. C., et al., Am J Physiol Gastrointest Liver Physiol 289, G1043-1051, 2005). It should be noted that the use of isofluorane anesthetic to carry out the present validation in mice may have resulted in a reduction in the rate of frequency of peristaltic contraction as such a effect has been reported by others (Torjman M C. et al., 2005).

TABLE 1

| | | | | | Frequency |
|---|---|---|---|---|---|
| | | | | | of |
| | Strain | | Imaging | Propagation | contractions |
| | (age) | Agents - Route | method | velocity | (/min) |
| Der-Silaphet, et al., | CD1 | 0.5 ml of Barium | X-ray | 1.6 ± 0.1 cm/s | 47 ± 1 |
| 1998 | mice | Fluid - gavaged | | (1.3-1.8 cm/s) | |
| Bercik, et al., | NIH | 0.2 ml of Iodine- | X-ray | Aborally: 1.1 ± | 44.1 ± 1.3 |
| 2004 | Swiss | based solution - | | 0.1 cm/s; orally: | |
| | mice | gavaged | | 0.4 ± 0.1 cm/s | |
| | (6-8 wk) | | | | |
| Der, et al., 2000 | C57BL/6 | 0.5 ml of Barium | X-ray | 1.3 cm/s | 45 ± 0.5 |
| | mice | Fluid - gavaged | | | |
| | (5-7 wk) | | | | |

In some embodiments, the present non-invasive NIR fluorescence imaging system in combination with injection of ICG can be used to image intestinal motility and can provide a method for diagnostic motility testing for intestinal motility disorders or dysfunction as well as the development and evaluation of therapeutic agents.

In some embodiments, the methods and compositions can be used to direct facilitate and evaluate treatments or therapies for gastrointestinal motility disorders. In some embodiments, the methods and compositions avoid the pitfalls of other methods, such as the difficulties of repeated oral contrast agent administration and the distension of the intestinal walls by the significant quantities of contrast agent required.

There are presently very few technologies with the ability to non-invasively image the gastrointestinal system in vivo and in real time. Consequently, there is continuing interest in non-invasive imaging methods and imaging agents for dynamically assessing gastrointestinal motility function in vivo in veterinary and human patients.

Novel methods and imaging agents for functional imaging of intestinal structure and function, motility in veterinary and human patients are described. Embodiments of the methods utilize highly sensitive optical imaging and fluorescent spectroscopy techniques to track or monitor packets of organic, soluble dyes being propelled through the gastrointestinal tract. The packets of organic dye may be tracked to provide quantitative information regarding propulsion, contractile rate and function. Thus, the disclosed methods provide non-invasive ways of assessing the gastrointestinal tract and its function.

Presently disclosed are methods and compositions for use in non-invasive imaging of intestinal contractile function with the simple administration of a NIR fluorescent dye that can be used to identify abnormal patterns and physiology, provide correct diagnosis of GI motility disorders or dysfunction, and guide evaluation of pharmacologic and surgical therapy for both veterinary and human patients.

In some embodiments a method of non-invasively assessing gastrointestinal motility in an individual, comprises administration of at least one imaging packet to the individual, the imaging packet containing ICG. In some embodiments, a method of measuring propulsion of the gastrointestinal system of a patient comprises at least one of contraction frequency and the propagation velocity.

In some other embodiments, the methods and compositions describe a method of identifying a patient suffering from or at risk of a gastrointestinal motility disorder. In some embodiments, the methods and compositions provide a method to accelerate both preclinical and clinical drug or therapy discovery, validation and approval by providing a convenient method for longitudinally assessing GI motility in veterinary or human patients. In some embodiments the methods and compositions described can be used to identify a gastrointestinal motility disorder in a patient. Thus, in some embodiments the methods and compositions described can be used to diagnose a gastrointestinal motility disorder in a patient. In some embodiments the methods and compositions described can be used to identify motility disorder, such as a blockage, in a particular portion of the intestinal tract of a veterinary or human patient.

In some embodiments, the methods and compositions provide a method to accelerate both preclinical and clinical drug or therapy discovery, validation and approval by providing a convenient method for longitudinally assessing GI motility in veterinary or human patients.

In some embodiments the methods and compositions described can be used to identify a gastrointestinal motility disorder in a patient. Thus, in some embodiments the methods and compositions described can be used to diagnose a gastrointestinal motility disorder in a patient. In some embodiments the methods and compositions described can be used to identify motility disorder, such as a blockage, in a particular portion of the intestinal tract of a veterinary or human patient.

In some embodiments the methods and compositions described can be used to identify a patient, at risk developing a gastrointestinal motility disorder. In some embodiments the methods and compositions described can be used to identify a particular portion of a patient's intestinal tract at risk of developing a gastrointestinal motility disorder. In some embodiments the methods and compositions described can be used to monitor a gastrointestinal motility disorder in a patient. In some embodiments the methods and compositions described can be used to direct therapy or treatment for a gastrointestinal motility disorder in a patient. In some embodiments, the methods and compositions described can be used to determine the efficacy of a therapy or treatment for a gastrointestinal motility disorder in a patient.

In some embodiments, a method of non-invasively imaging gastrointestinal motility in an animal, the method comprising: a) administering to said animal at least one packet containing a fluorescent agent or foodstuff containing at least one auto fluorescent compound; b) allowing sufficient time for said at least one packet containing a fluorescent agent to be processed by the liver and excreted in bile or the foodstuff to enter into the gastrointestinal tract; c) illuminating the abdominal tissue surface with an excitation light to excite the fluorescent or auto fluorescent agent; d) continuously, non-invasively detecting fluorescent or auto fluorescent emissions from the fluorescent agent to image at least a region of the intestinal tract; (e) capturing a plurality of images of said one or more regions of the intestinal tract, based on said detected fluorescent images; and (f) tracking in vivo propulsion of said at least one packet or said auto fluorescent compound through the one or more regions of the intestinal tract, based on said captured images.

In some embodiments, a method of non-invasively imaging gastrointestinal motility in an animal, the method comprising: a) administering to said animal at least one packet containing a indocyanine green (“ICG”) as a fluorescent agent; b) allowing sufficient time for said at least one packet containing a ICG to be processed by the liver and excreted in bile; c) illuminating the abdominal tissue surface with an excitation light to excite the ICG; d) continuously, non-invasively detecting fluorescent emissions from the ICG fluorescent agent to image at least a region of the intestinal tract; (e) capturing a plurality of images of said one or more regions of the intestinal tract, based on said detected fluorescent images; and (f) tracking in vivo propulsion of said at least one packet through the one or more regions of the intestinal tract, based on said captured images.

In some embodiments, the fluorescent agent is applied by intradermal injection. In some embodiments, the fluorescent agent is applied intravenously. In some embodiments, the fluorescent or auto fluorescent agent has an excitation wavelength ranging from about 700 nm to about 900 nm. In some embodiments, step (d) comprises using an intensified charge-coupled camera. In some embodiments, step (c) comprises illuminating the tissue surface with an excitation light source selected from group consisting of laser diodes, semiconductor laser diodes, gas lasers, light emitting diodes, and combinations thereof.

In some embodiments, a method wherein, capturing said plurality of images comprises a camera integration time in the range of about 10 milliseconds to about 1 second. In some embodiments, a method wherein, capturing said plurality of images comprises a camera integration time in the range of about 100 milliseconds to about 800 milliseconds. In some embodiments, a method wherein, capturing said plurality of images comprises a camera integration time greater than 200 milliseconds. In some embodiments, a method wherein, capturing said plurality of images comprises a camera integration time greater than 200 milliseconds. In some embodiments, a method further comprising calculating intestinal flow velocity to assess intestinal functionality. In some embodiments, a method further comprising calculating intestinal peristaltic wave rate to assess intestinal functionality. In some embodiments, a method further comprising calculating intestinal segmental contraction rate to assess intestinal functionality.

In some embodiments, a method of non-invasively imaging gastrointestinal motility in an animal, the method comprising: (a) administering to the animal at least one packet containing a fluorescent agent that when administered in vivo is excreted in bile; (b) allowing sufficient time for the at least one packet containing a fluorescent agent to be processed by the liver and excreted in bile; (c) illuminating the abdominal tissue surface with an excitation light to excite the fluorescent agent; (d) continuously, non-invasively detecting fluorescent emissions from the fluorescent agent to image at least a region of the intestinal tract; (e) capturing a plurality of images of the one or more regions of the intestinal tract, based on the detected fluorescent images; and (f) tracking in vivo propulsion of the at least one packet through the one or more regions of the intestinal tract, based on the captured images. In some embodiments, the fluorescent agent comprises indocyanine green (“ICG”). In some embodiments, the method comprises injecting the fluorescent agent intradermally. In some embodiments, the method comprises administering the fluorescent agent intravenously. In some embodiments, the fluorescent agent has an excitation wavelength ranging from about 700 nm to about 900 nm. In some embodiments, the method comprises using an intensified charge-coupled camera. In some embodiments, the method comprises illuminating the tissue surface with an excitation light source selected from group consisting of laser diodes, semiconductor laser diodes, gas lasers, light emitting diodes, and combinations thereof.

In some embodiments, a method of non-invasively assessing gastrointestinal motility in an individual is described. In some embodiments, a method of non-invasively measuring propulsion of the gastrointestinal system of a patient comprises at least one of contraction frequency and the propagation velocity, is described. In some embodiments, a method of non-invasively identifying a patient suffering from or at risk of a gastrointestinal motility disorder is described.

In some embodiments, a method of non-invasively identifying a patient in need of therapy for a gastrointestinal motility disorder is described.

In some embodiments, a method of non-invasively identifying a patient in need of surgery for a gastrointestinal blockage is described. In some embodiments, a method of accelerating both preclinical and clinical drug or therapy discovery, validation and approval by providing a convenient method for longitudinally assessing gastrointestinal motility in veterinary or human patients is described. In some embodiments, a method of non-invasively identifying the portion of a patient's intestinal tract at risk of developing a gastrointestinal motility disorder is described. In some embodiments, a method of non-invasively identifying the particular portion of a patient's intestinal tract in need of therapy due to a gastrointestinal motility disorder is described. In some embodiments, a method of non-invasively identifying the particular portion of a patient's intestinal tract in need of surgery due to a gastrointestinal motility disorder is described. In some embodiments, a method of non-invasively determining the efficacy of a therapy for a gastrointestinal motility disorder in a patient being treated for a gastrointestinal motility disorder is described. In some embodiments, a method of non-invasively monitoring the progress of a patient being treated for a gastrointestinal motility is described. In some embodiments, the method comprises capturing the plurality of images comprising a camera integration time in the range of about 10 milliseconds to about 1 second. In some embodiments, the described method includes capturing the plurality of images comprising a camera integration time in the range of about 100 milliseconds to about 800 milliseconds. In some embodiments, the described method includes capturing the plurality of images comprising a camera integration time greater than 200 milliseconds. In some embodiments, the described method includes calculating intestinal flow velocity to assess intestinal functionality. In some embodiments, the described method includes calculating intestinal peristaltic wave rate to assess intestinal functionality. In some embodiments, the described method includes calculating intestinal segmental contraction rate to assess intestinal functionality.

In some embodiments, a method wherein capturing the plurality of images comprises a camera integration time in the range of about 100 milliseconds to about 800 milliseconds.

In some embodiments, a method wherein capturing the plurality of images comprises a camera integration time greater than 200 milliseconds. In some embodiments, a method of calculating intestinal flow velocity is used to assess intestinal functionality. In some embodiments, a method of calculating intestinal peristaltic wave rate is used to assess intestinal functionality. In some embodiments, a method of calculating intestinal segmental contraction rate is used to assess intestinal functionality.

In some embodiments, a method of non-invasively assessing gastrointestinal function in an individual, comprising: (a) administering to the animal at least one packet containing a fluorescent agent having a characteristic excitation wavelength and a characteristic fluorescence emission wavelength and that when administered in vivo is excreted in bile or foodstuff containing at least one auto fluorescent compound having a characteristic excitation wavelength and a characteristic fluorescence emission wavelength; (b) allowing sufficient time for the at least one packet containing the fluorescent agent to be processed and excreted in bile or enter the intestinal tract; (c) noninvasively illuminating a tissue surface of the intestinal tract on the individual's body with radiation at the characteristic excitation wavelength; (d) noninvasively detecting fluorescence emissions from the agent within a region of interest and capturing a plurality of fluorescence images for an interval of time; (e) using the fluorescence images to visualize gastrointestinal motion in a region of interest and to track the location of the agent in the region of interest as a function of time to obtain a set of tracked image locations as a function of time; (f) determining from the tracked locations as a function of time an initial intestinal propulsion measurement; (g) optionally, comparing the initial intestinal propulsion measurement to a subsequently determined intestinal propulsion measurement; and (h) determining from the results of d) and e), and f) if present, the functionality of a gastrointestinal tract in the region of interest in the individual.

In some embodiments, the fluorescent agent comprises indocyanine green (“ICG”). In some embodiments are included a method wherein tracking the location of the agent includes capturing each image at an integration time ranging from about 10 ms to about 1 s. In some embodiments, a method wherein the characteristic excitation wavelength is in the region of 750-800 nm, and the characteristic fluorescence emission wavelength is greater than 800 nm. In some embodiments are included a method wherein the intestinal propulsion measurement comprises at least one of intestinal peristaltic wave rate and segmental contraction rate. In some embodiments, a method wherein the initial intestinal propulsion measurement comprises an initial intestinal peristaltic wave rate; is performed and comprises comparing the initial intestinal peristaltic wave rate to a subsequently determined intestinal peristaltic wave rate; and the determining the functionality of a gastrointestinal structure includes determining that intestinal function in the region of interest is improved if the subsequent intestinal peristaltic wave rate is greater than the initial intestinal peristaltic wave rate.

In some embodiments, a method wherein the initial intestinal propulsion measurement comprises an initial intestinal segmental contraction rate; is performed and comprises comparing the initial intestinal segmental contraction rate to a subsequently determined intestinal segmental contraction rate; and the determining the functionality of a gastrointestinal structure includes determining that intestinal function in the region of interest is improved if the subsequent intestinal segmental contraction rate is greater than the initial intestinal segmental contraction rate.

In some embodiments, a method comprising, administering to the individual a treatment to ameliorate the symptoms of a gastrointestinal disease or disorder is described. In some embodiments, a method wherein a intestinal propulsion measurement of less than a control value indicates the presence of a gastrointestinal disease or disorder is described. In some embodiments, a method of non-invasively identifying a patient suffering from or at risk of a gastrointestinal motility disorder is described. In some embodiments, a method of non-invasively identifying a patient at risk of a gastrointestinal motility disorder, is described. In some embodiments, a method of non-invasively identifying a patient in need of therapy for a gastrointestinal motility disorder is described. In some embodiments, a method of non-invasively identifying a patient in need of surgery for a gastrointestinal blockage is described. In some embodiments, a method of accelerating both preclinical and clinical drug or therapy discovery, validation and approval by providing a convenient method for longitudinally assessing gastrointestinal motility in veterinary or human patients is described. In some embodiments, a method of non-invasively identifying the portion of a patient's intestinal tract at risk of developing a gastrointestinal motility disorder is described. In some embodiments, a method of non-invasively identifying the particular portion of a patient's intestinal tract in need of therapy due to a gastrointestinal motility disorder is described. In some embodiments, a method of non-invasively identifying the particular portion of a patient's intestinal tract in need of surgery due to a gastrointestinal motility disorder is described. In some embodiments, a method of non-invasively determining the efficacy of a therapy for a gastrointestinal motility disorder in a patient being treated for a gastrointestinal motility disorder is described. In some embodiments, a method of non-invasively monitoring the progress of a patient being treated for a gastrointestinal motility, utilizing the methods of claims is described. In some embodiments, the patient, subject or individual is an animal. In some embodiments, the patient, subject or individual is a mammal. In some embodiments, the patient, subject or individual is a human.

The methods and compositions disclosed are applicable to all animals that are subject to gastrointestinal motility diseases and disorders but have particular utility in companion animals and humans.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Using Auto Fluorescence to Non-Invasively Monitor Intestinal Motions

Six to eight week old mice that happened to carry a Rasa 1 floxed allele (rasa 1 fl/fl), i.e. the Rasa 1 allele is flanked by a Lox P sites to facilitate removal). Rasa I fl/fl mice are completely normal (until exposed to Cre-recombinase) with regards to GI function and were maintained in standard cages with free access to water and pellets (Purina 5053, Labdiet, PMI Nutritional International, St. Louis, Mo., USA) prior to imaging. Once anesthetized with isofluorane, the hair of the mice was clipped and depilatory agents were used to remove residual hair 24 hr prior to fluorescence imaging. Mice were housed and maintained in a pathogen-free mouse facility accredited by the American Association for Laboratory Animal Care. All experiments were performed in accordance with the guidelines of the Institutional Animal Care and Use Committee. Animal experiments were approved by University of Texas Health Science Center Institutional Animal Care and Usage Committee in accordance with NIH guidelines.

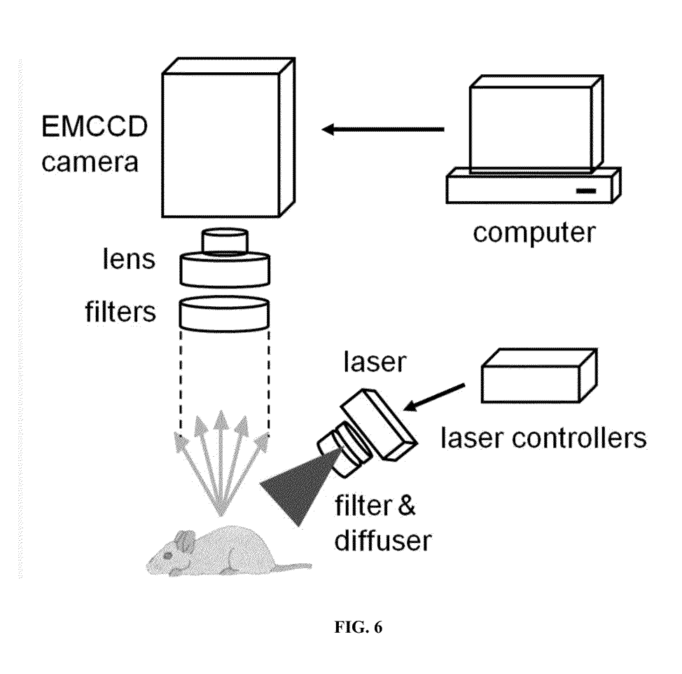

In order to utilize auto fluorescence from foodstuffs for NIR imaging, dynamic fluorescence images were acquired for a period of approximately 6 min using a custom-built NIR fluorescence imaging system, the basics of which are presented in FIG. 6 and has been described elsewhere (for example U.S. Pat. Nos. 8,084,753; 7,875,260; 7,865,230; 7,599,732; 7,328,059; 7,268,873; 7,187,441; 7,054,002; 6,930,777; 6,771,370; 5,818,583; 5,865,754 and Kwon and Sevick-Muraca, Lymphat Res Biol 5, 219-231, 2007; Kwon and Sevick-Muraca, J Immunol Methods 360, 167-172, 2010). 660 nm light from a laser diode (500 mA, 660 nm) via a diffuser and a 660 nm bandpass filter (Image quality, model 660.0-1.0, Andover Corp., Salem, N.H.) was used to illuminate the whole body of an anesthetized mouse. Throughout the process the body temperature of the mice was maintained by placing them on a 37° C. warming pad. Red fluorescence occurring in the abdominal region of the mouse was collected through a 710 nm bandpass filter (Image quality, model 710.0-2.0, Andover Corp., Salem, N.H.). Fluorescent images were acquired using an electron-software (Digital Optics, Aukland, New Zealand).

Necropsy of a mouse with exposure of the abdomen was also performed and the GI tract was removed and laid out on black paper to more clearly detail the auto fluorescence imaging. Animal experiments were approved by and were done in accordance with The University of Texas Health Science Center Institutional Animal Care and Usage Committee and in accordance with NIH guidelines.

Fluorescent imaging data were analyzed using Matlab (The MathWorks, Inc., Natick, Mass., USA). Regions of interest (ROIs) of equivalent areas were selected along the fluorescent intestines on sequential frames of fluorescence images as was similarly reported for lymphatic imaging (see for example, Kwon S and Sevick-Muraca E M, Neurogastroenterol Motil, 23:881-e344, 2011). Small circular ROls were selected of equivalent size to minimize the respiratory motion artifacts. The averaged fluorescent intensity within each selected ROI was plotted as a function of imaging time and ROI to generate a three-dimensional (3-D) spatio-temporal map. Thus, the propagation velocity and the frequency of intestinal contractility were assessed. Peristaltic motion (or contractility) was defined as aboral propagation of contractile waves along the intestines.

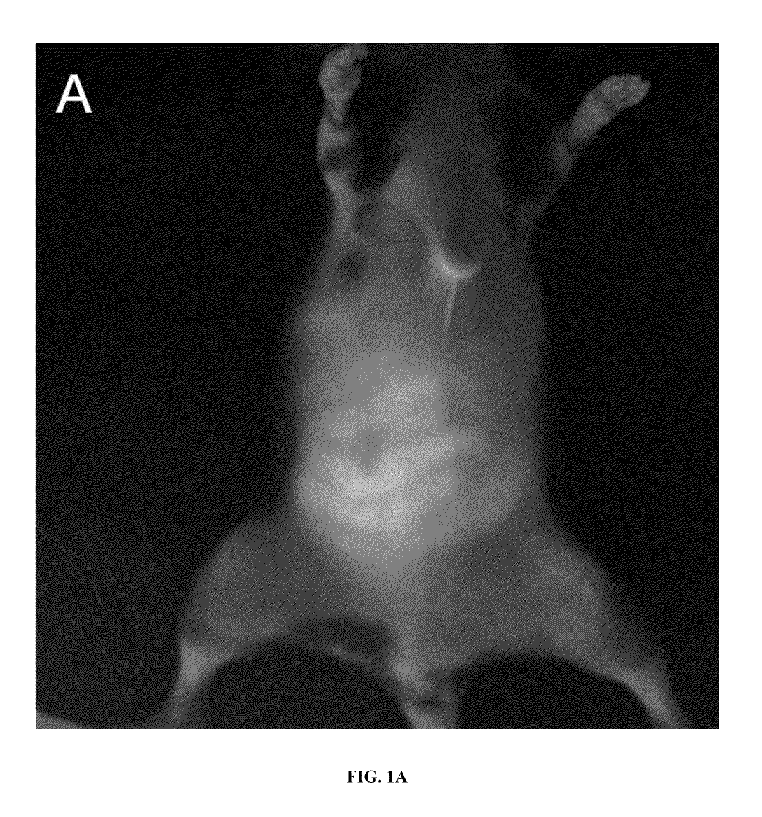

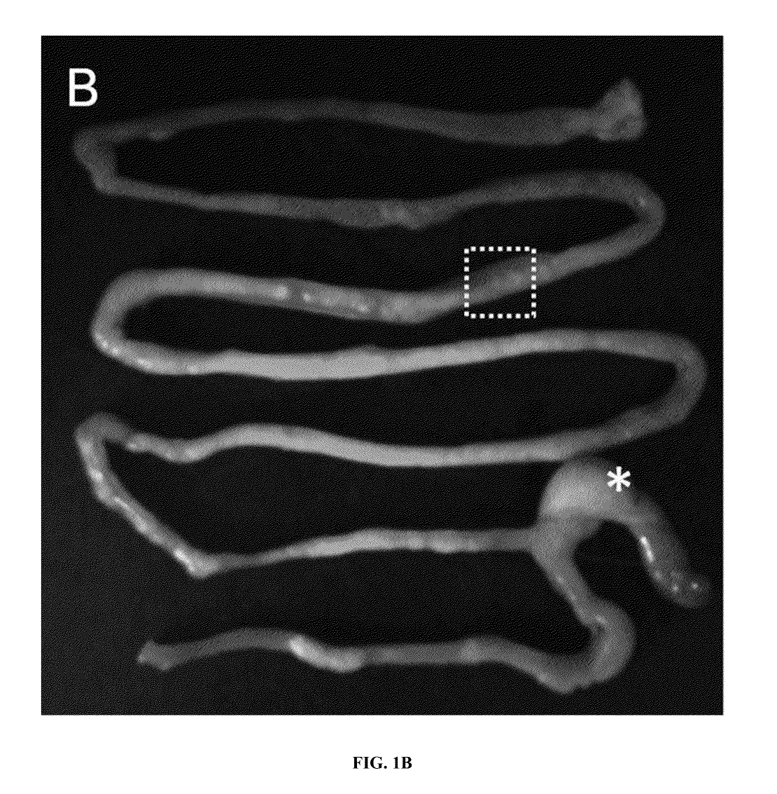

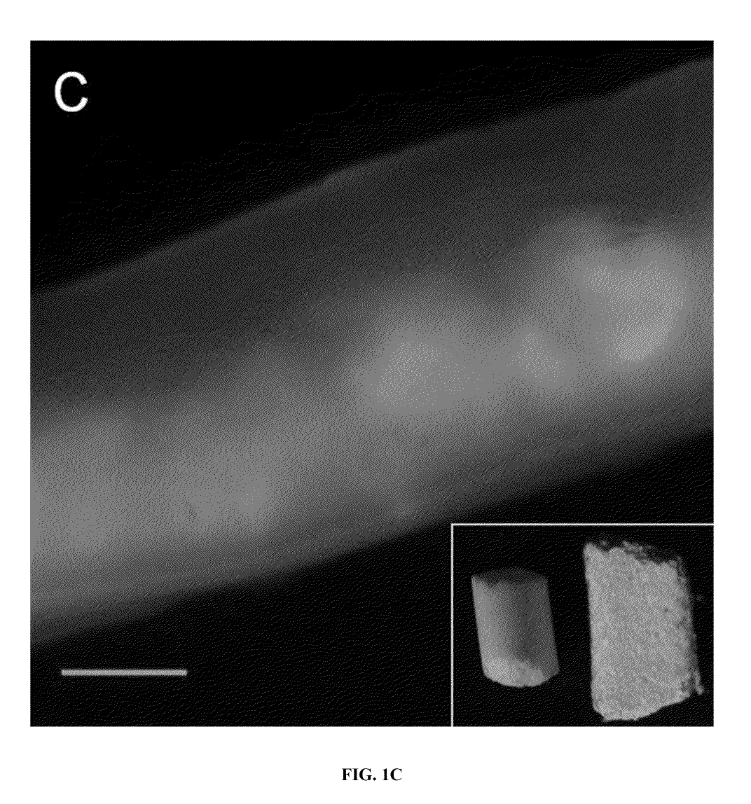

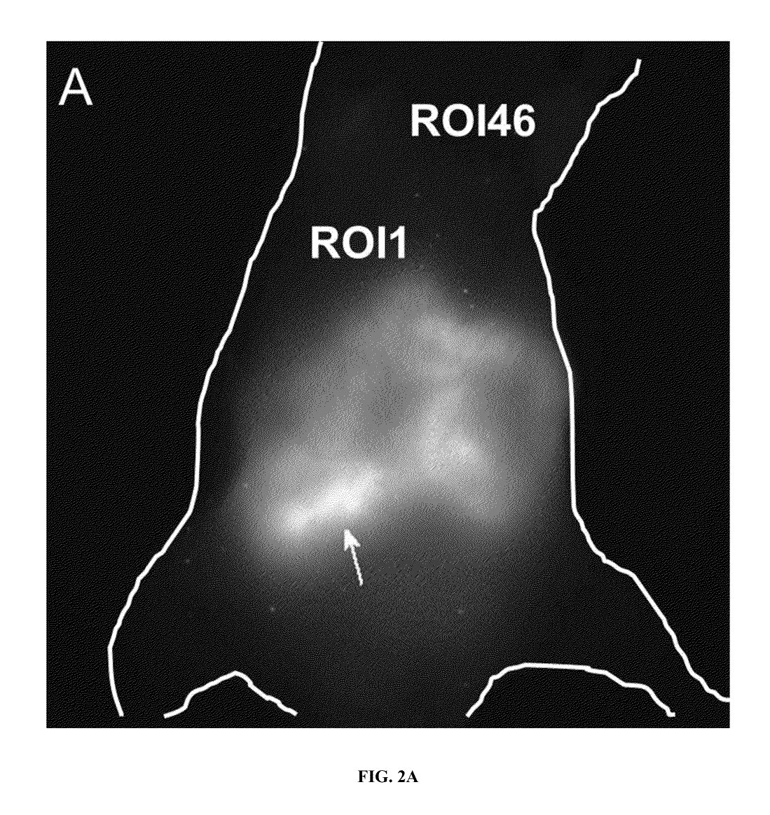

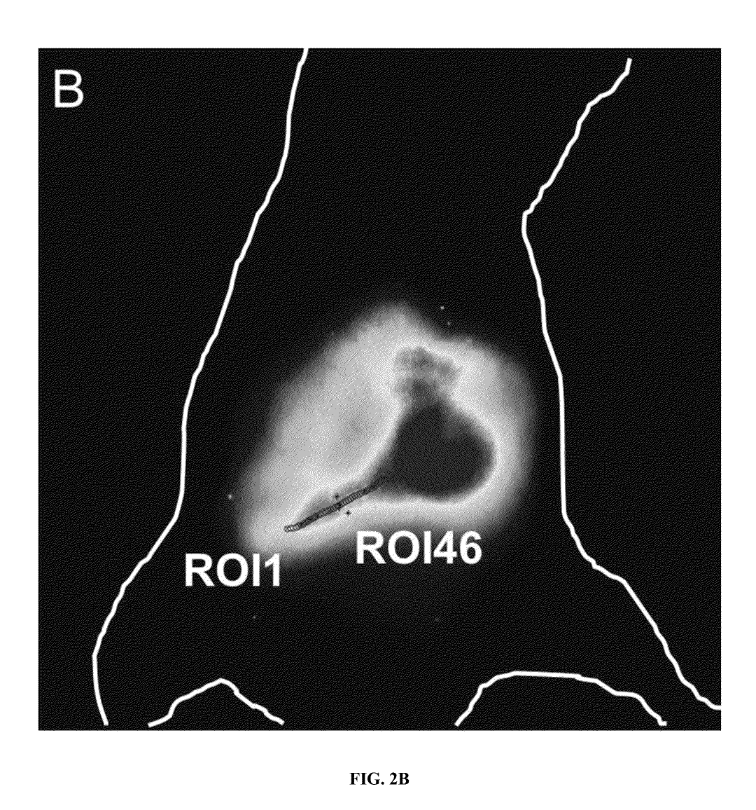

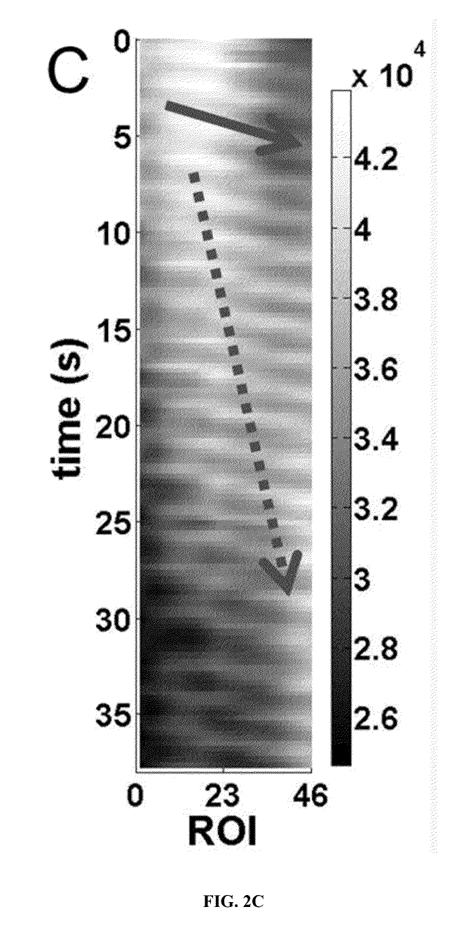

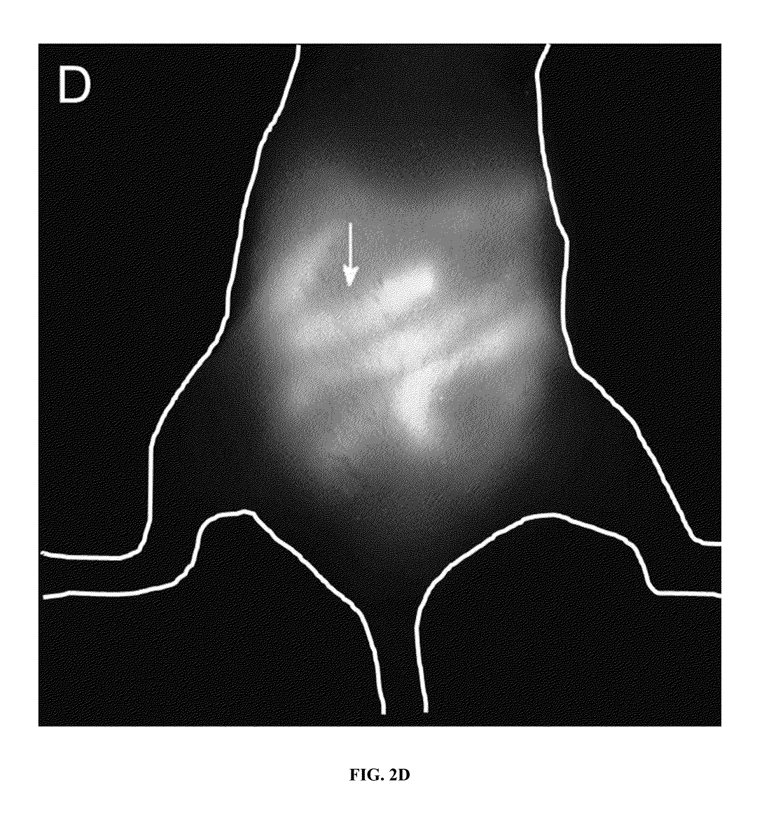

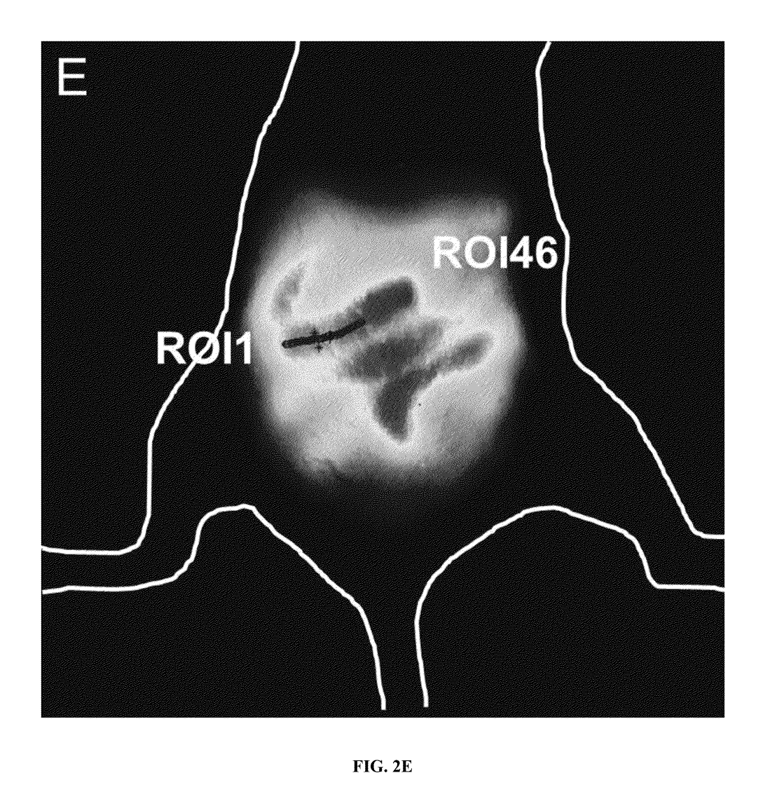

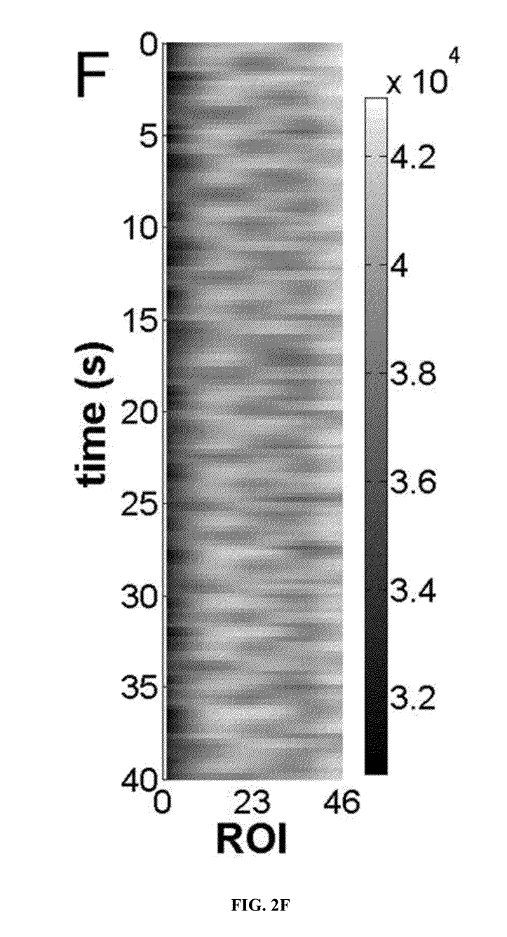

Auto fluorescence along the GI tract in the abdominal region of the mice was observed (shown in FIG. 1A). Mice showed different digestive status at each imaging time point as indicated by auto fluorescence imaging. Thus, non-uniformly distributed auto fluorescence was detected throughout the intestines in the excised whole GI tract (shown in FIG. 1B) and a magnified fluorescence image demonstrating that auto fluorescence came from ingested food containing chlorophyll (FIG. 1C). Since in vivo auto fluorescence images showed clear delineation of the intestines in the abdomen, we performed dynamic auto fluorescence imaging. Peristaltic motion, the radially symmetrical contraction and relaxation of smooth muscle to propel contents though the intestines, was imaged non-invasively using video images. To quantify peristaltic motion, 46 ROls were selected along the fluorescent intestines (shown in FIG. 2B) and a 3-D map was generated as a function of time and ROI (shown in FIG. 2C). A 3-D map showed the aboral propagation of the contractile wave along the small intestine. The patterns illustrated in FIG. 2C are similar to those during the migrating motor complexes eMMC), due to slow movement of fluorescent intraluminal contents (red broken arrow). The frequency of peristalsis and propagation velocity were measured in sections below the one analyzed. Although no corrections for respiratory movements were applied in the construction of 3-D spatio-temporal maps, dynamic changes of fluorescent intensities of small ROIs situated in the center of the fluorescent intestines are shown in FIGS. 2D and 2E and arise due to intestinal contractile activity.

Thus the ability to non-invasively and quantitatively image intestinal motions using only auto fluorescence of compounds present within foodstuffs. This method did not require the administration of an exogenous imaging agent.

The fluorescence imaging system described can easily be combined with a traditional, invasive video imaging method. As intestinal contractile motions are influenced by pathological conditions (symptoms related to gastrointestinal diseases and disorders), this simple, imaging technique using auto fluorescence can be used as, among other things, to monitor disease progression and possibly therapeutic response.

### Example 2

### Using Exogenous Fluorescence to Monitor Intestinal Motions

In order to clearly characterize and demonstrate the abilities of the methods disclosed, studies were carried out on mice as a model for all animals, including but not limited to those animals that can suffer a gastrointestinal motility disease or disorder. Such animals include, but are not limited to, laboratory animals, domesticated and livestock animals, wild and captured animals such as those that are in zoos, companion animals and humans.

Mice were chosen because part of the study involved opening the peritoneal cavity and removing organs to provide detailed information about the method and its abilities. Therefore, the subjects of the study were Female C57BL6 mice (4-6 wk old; Charles River, Wilmington, Mass., USA) that were housed and maintained in a pathogen-free mouse facility accredited by the American Association for Laboratory Animal Care. All studies were performed in accordance with the guidelines of the Institutional Animal Care and Use Committee. Animal experiments were approved by University of Texas Health Science Center Institutional Animal Care and Usage Committee in accordance with NIH guidelines.

In order to carry out in vivo dynamic NIR imaging of GI Motility, the mice were anesthetized with isofluorane and maintained at 37° C. on a warming pad. A volume of 10 μl and 50 ul of 645 μM of IC-Green (Akorn, Inc. Buffalo Grove, Ill., USA) dissolved in mixture of distilled water and 0.9% Sodium Chloride in a volume ratio of 1:9 was injected intradermally and intravenously, respectively.

For intravenous injection of ICG, a catheter was placed in the tail vein of anesthetized mice to facilitate the subsequent delivery of the fluorescent dye. Dynamic fluorescence images were acquired for a period of approximately 6 min immediately before and up to 24 hrs after intravenous injection using a custom-built NIR fluorescence imaging system, the basics of which are presented in FIG. 6 and has been described elsewhere (for example U.S. Pat. Nos. 8,084,753; 7,875,260; 7,865,230; 7,599,732; 7,328,059; 7,268,873; 7,187,441; 7,054,002; 6,930,777; 6,771,370; 5,818,583; 5,865,754 and Kwon and Sevick-Muraca, Lymphat Res Biol 5, 219-231, 2007; Kwon and Sevick-Muraca, J Immunol Methods 360, 167-172, 2010). Anesthetized mice were uniformly illuminated by 785 nm light from a laser diode and the emitted fluorescence was collected using an Electron Multiplying Charge Coupled Device (EMCCD) camera with a holographic filter and a bandpass filter placed prior to a 28 mm lens. Image acquisition with the camera exposure time of 100 ms for fluorescence and white light imaging was accomplished by V++ software. The abdominal skin temperature was monitored for temperature changes during dynamic fluorescence imaging using an infrared thermometer but no temperature changes were observed. Because it was possible that the non-invasive imaging of the intestines may not distinguish the GI lumen from blood and lymphatic vessels, necropsy of the mouse with exposure of the abdomen was performed at 1 hr post injection (p.i.) and the GI tract was removed and laid out on a black paper for intravital imaging.

As expected, intravenous and intradermal administrations resulted in different clearance rates. When ICG was injected intravenously, the fluorescent signal in the liver was greatly reduced at 1 h post injection, whereas fluorescence was still high in the liver and gallbladder after intradermal administration, due to continuous lymph formation after intradermal injection, clearance back to blood circulation via subclavian vein, and finally excretion by the liver into bile. Regardless of the route of injection ICG finds its way to the liver and is excreted into bile.

The data obtained were analyzed with a custom Matlab (The MathWorks, Inc., Natick, Mass., USA) program and ImageJ (National Institutes of Health, Bethesda, Md., USA). Fixed regions of interest (ROIs) from equivalent areas were selected along the length of the fluorescent intestine. An average fluorescent intensity within each ROI was plotted as a function of imaging time and/or length of the intestine. A three-dimensional plot of fluorescent intensity as a function of time and distance was generated and used as a spatio-temporal map to assess the propagation velocity and the frequency of the propagation along the intestine. No corrections were made for respiratory movements in the construction of the three-dimensional spatio-temporal maps. The packet of fluorescent contents is represented by the peak in fluorescence intensity which propagates along the intestine with time. Two ROIs were identified, from which the distance and time information was extracted to measure the propagation velocity. In this study, peristalsis was defined as aboral propagation of contraction waves along the intestine.

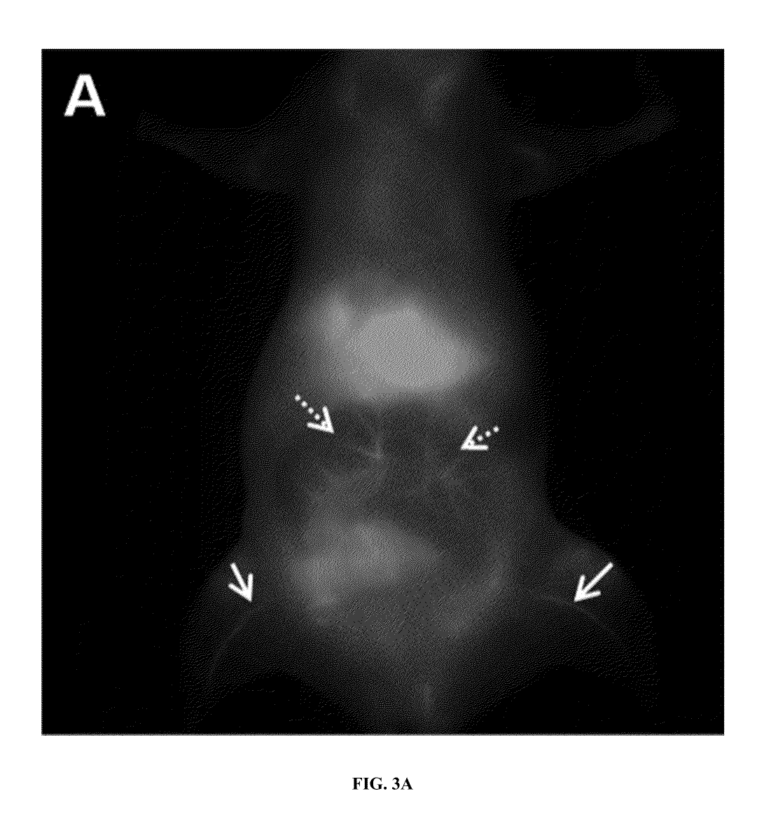

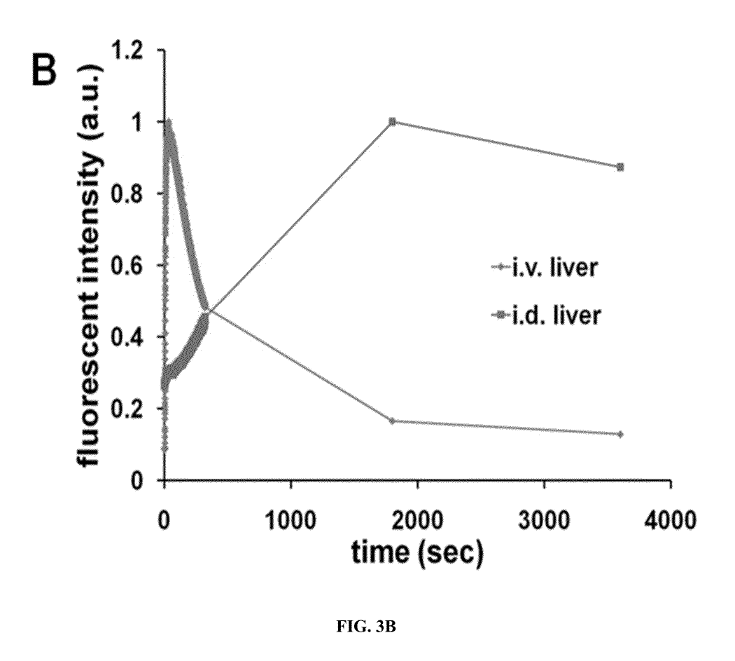

Following the intravenous injection of ICG, rapid distribution of the ICG throughout the mouse was observed within seconds. Fluorescent signal was detected in the chest, blood vasculature, and subsequently the liver. FIG. 3A illustrates a fluorescent image showing blood vessels in the skin and gut at 4 sec post intravenous administration of ICG. Fluorescent vessels could be visualized for up to 2 min after intravenous administration.

Similar results were obtained with both intravenous and intradermal administration of ICG but the route of administration resulted in different clearance rates as illustrated in FIG. 3B which is a fluorescent intensity plot as a function of time. Furthermore, when ICG was injected intravenously, the fluorescent signal in the liver was greatly reduced at 1 hr post injection, whereas fluorescence was still high in the liver following intradermal administration.

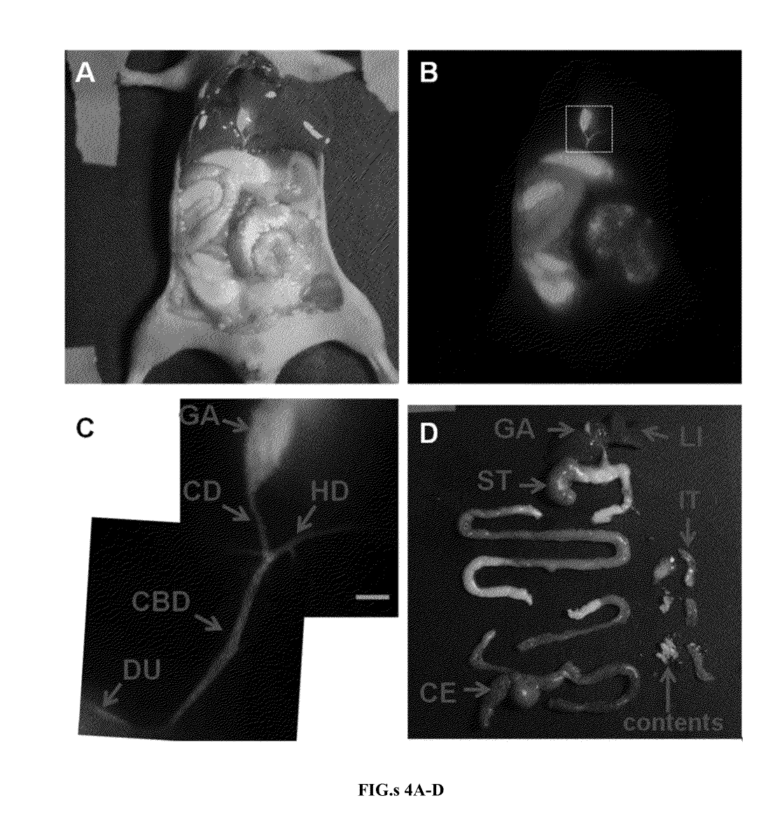

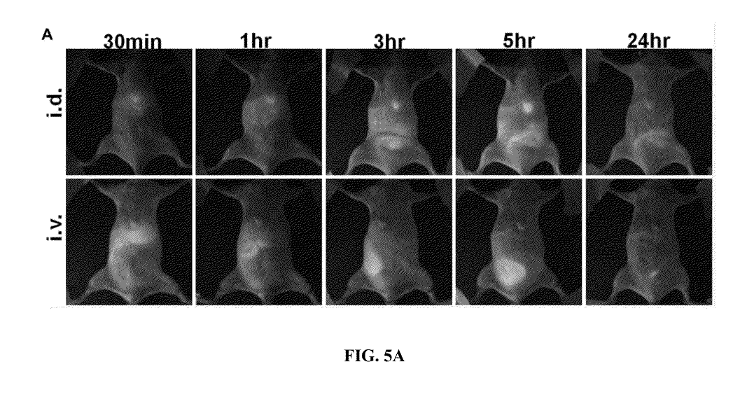

An overlay of white light and fluorescent images obtained at 1 h post injection but prior to the opening of the peritoneal cavity of a mouse is shown in FIG. 4A fluorescent signal in the biliary system and there is fluorescence in the intestines. An overlay of white light and fluorescent images of the mouse following exposure of the abdomen at 1 h post injection is shown in FIG. 4B where the organs and tissues involved can be more clearly identified. FIG. 4C is a magnified fluorescent image of the rectangle in FIG. 4A showing the biliary system. (Scale bar=1 mm; CBD: common bile duct; CD: cystic duct; DU: duodenum; GA: gallbladder; HD: hepatic duct). At 1 hr post injection there was strong but non-uniform fluorescence was detected throughout the small intestine with weak fluorescence observed in the cecum. Fluorescent signal arising from the stomach was absent at all times.

To more clearly identify the organs and tissues that fluorescence, the GI tract was removed from the body and the tissues isolated (FIG. 4D: CBD: common bile duct; CD: cystic duct; CE: cecum; DU: duodenum; GA: gallbladder; HD: hepatic duct; IT: intestinal tissues; LI: liver). The small intestine was cut into three segments and the contents were removed. Analysis of dissected small intestine and its contents revealed that the fluorescent signal does not arise from the intestinal tissues, but rather from its contents.

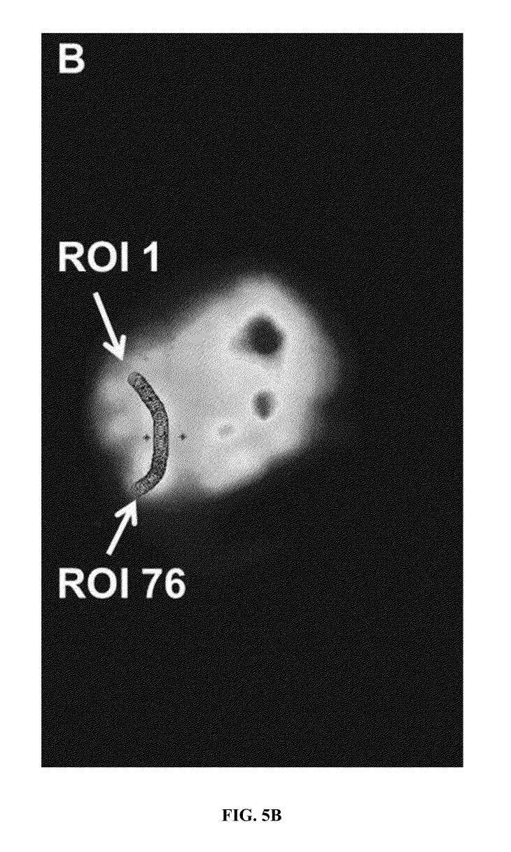

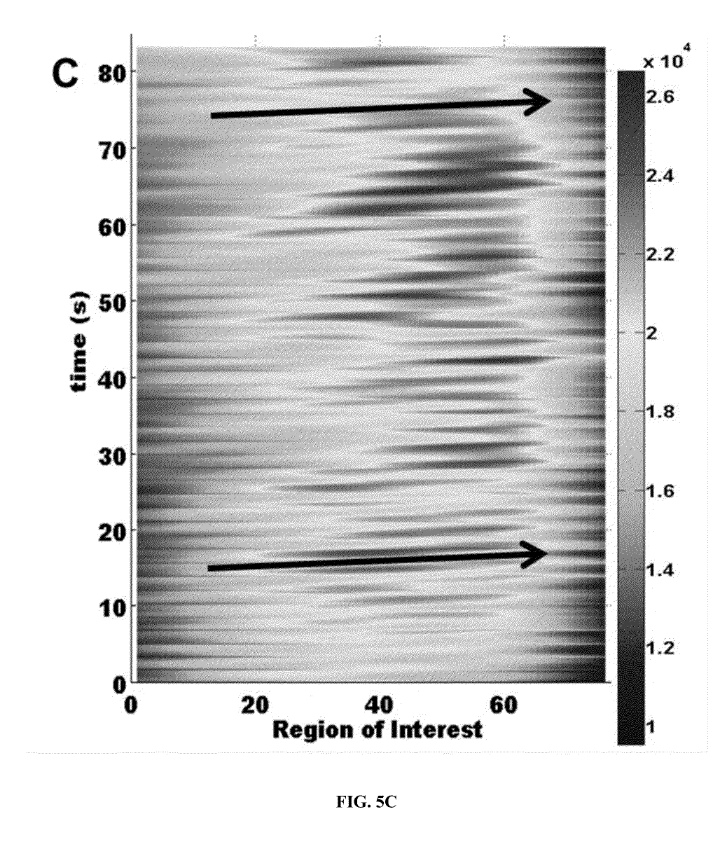

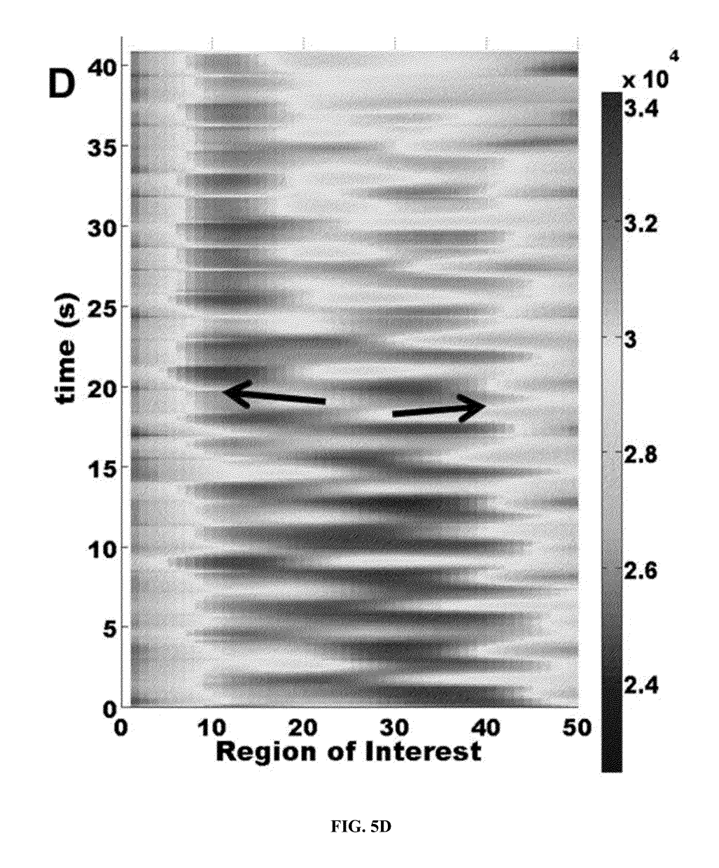

FIG. 5A represents selected frames from a real-time image series taken of a mouse after intradermal and intravenous injection of ICG, showing intestinal transit of ICG at different times after administration.

Video imaging of a mouse after intradermal injection demonstrated the contractile function of the small intestine 30 min after injection, and showed secretion of ICG-laden bile into the duodenum. Vigorous contractility of the fluorescent small intestine was observed 1 hr and 3 hr after intradermal injection. Fluorescent signal in the cecum was detected as early as 1 hr after injection and also observed at 24 hr post injection as shown in FIG. 5.

To determine the contractility along the length of the small intestine, a series of regions of interest (ROIs) were selected on the static images similar to the analysis conducted in dynamic X-ray fluoroscopy. FIG. 5A depicts an example of the fluorescent image, where 76 ROIs were selected on the fluorescent small intestine in the same animal. FIG. 5B shows the spatial-temporal map of fluorescent intensity denoted in color with x-axis detailing position along the intestine and y-axis the time of imaging. The maximum intensity signifies “waves” of ICG transit, demonstrating the aboral propagation of the contractile wave (solid arrow) along the small intestine at the propagation velocity of 2.04±1.15 cm/s and 27 cycles/min in the mouse. The dotted arrow in FIG. 5B signifies combined peristaltic contraction and segmental motion where motion of ICG in the intestine occurs in opposite directions for a short distance. The frequency of peristalsis and propagation velocity in the duodenum was 35 cycles/min and 0.82±0.5 cm/s, respectively. FIG. 5C shows another example of the spatial-temporal map of fluorescent intensity along the small intestine, demonstrating segmental contractions. Peristaltic waves in the cecum occurred at the frequency of 0.52 cycles/min and at the propagation velocity of 0.34±0.06 cm/s. Vigorous segmental motions moving contents back and forth with the small intestine 1 hr after intravenous injection were also observed. While the described embodiments illustrate use of the present compositions and methods on mice, those of skill in the art would readily recognize that these methods and compositions could be readily applied to both veterinary and human medicine.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Huizing a, J. D., Thuneberg, L., Vanderwinden, J. M. and Rumessen, J. J., “Interstitial cells of Cajal as targets for pharmacological intervention in gastrointestinal motor disorders,” Trends Pharmacol Sci 18, 393-403 (1997).
Huizing a, J. D., Thuneberg, L., Kluppel, M., Malysz, J., Mikkelsen, H. B. and Bernstein, A., “W/kit gene required for interstitial cells of Cajal and for intestinal pacemaker activity,” Nature 373, 347-349 (1995).
Bratten, J. R. and Jones, M. P., “Small intestinal motility,” Curr Opin Gastroenterol 23, 127-133 (2007).
Husebye, E., “The patterns of small bowel motility: physiology and implications in organic disease and functional disorders,” Neurogastroenterol Motil 11, 141-161 (1999).
Seerden, T. C., Lammers, W. J., De Winter, B. Y., De Man, J. G. and Pelckmans, P. A., “Spatiotemporal electrical and motility mapping of distension-induced propagating oscillations in the murine small intestine,” Am J Physiol Gastrointest Liver Physiol 289, G1043-1051 (2005).
Hennig, G. W., Costa, M., Chen, B. N. and Brookes, S. J., “Quantitative analysis of peristalsis in the guinea-pig small intestine using spatio-temporal maps,” J Physiol 517 (Pt 2), 575-590 (1999).
Huizing a, J. D., Ambrous, K. and Der-Silaphet, T., “Co-operation between neural and myogenic mechanisms in the control of distension-induced peristalsis in the mouse small intestine,” J Physiol 506 (Pt 3), 843-856 (1998).
Grivel, M. L. and Ruckebusch, Y., “The propagation of segmental contractions along the small intestine,” J Physiol 227, 611-625 (1972).
Der-Silaphet, T., Malysz, J., Hagel, S., Larry Arsenault, A. and Huizing a, J. D., “Interstitial cells of cajal direct normal propulsive contractile activity in the mouse small intestine,” Gastroenterology 114, 724-736 (1998).
Der, T., Bercik, P., Donnelly, G., Jackson, T., Berezin, I., Collins, S. M. and Huizing a, J. D., “Interstitial cells of cajal and inflammation-induced motor dysfunction in the mouse small intestine,” Gastroenterology 119, 1590-1599 (2000).
Bercik, P., Wang, L., Verdu, E. F., Mao, Y. K., Blennerhassett, P., Khan, W. I., Kean, I., Tougas, G. and Collins, S. M., “Visceral hyperalgesia and intestinal dysmotility in a mouse model of postinfective gut dysfunction,” Gastroenterology 127, 179-187 (2004).
Kwon, S. and Sevick-Muraca, E. M., “Noninvasive quantitative imaging of lymph function in mice,” Lymphat Res Biol 5, 219-231 (2007).
Kwon, S. and Sevick-Muraca, E. M., “Functional lymphatic imaging in tumor-bearing mice,” J Immunol Methods 360, 167-172 (2010).
Sevick-Muraca, E. M., Sharma, R., Rasmussen, J. C., Marshall, M. V., Wendt, J. A., Pham, H. Q., Bonefas, E., Houston, J. P., Sampath, L., Adams, K. E., Blanchard, D. K., Fisher, R. E., Chiang, S. B., Elledge, R. and Mawad, M. E., “Imaging of lymph flow in breast cancer patients after microdose administration of a near-infrared fluorophore: feasibility study,” Radiology 246, 734-741 (2008).
Rasmussen, J. C., Tan, I. C., Marshall, M. V., Adams, K. E., Kwon, S., Fife, C. E., Maus, E. A., Smith, L. A., Covington, K. R. and Sevick-Muraca, E. M., “Human Lymphatic Architecture and Dynamic Transport Imaged Using Near-infrared Fluorescence,” Transl Oncol 3, 362-372 (2010).
Maus, E. A., Tan, I. C., Rasmussen, J. C., Marshall, M. V., Fife, C. E., Smith, L. A., Guilliod, R. and Sevick-Muraca, E. M., “Near-infrared fluorescence imaging of lymphatics in head and neck lymphedema,” Head Neck (2010).
Tan, I. C., Maus, E. A., Rasmussen, J. C., Marshall, M. V., Adams, K. E., Fife, C. E., Smith, L. A. and Sevick-Muraca, E. M., “Near-infrared fluorescence assessment of contractile function following manual lymphatic drainage,” Archives of Physical Medicine and Rehabiliation, Accepted for publication, (2010).
Cherrick, G. R., Stein, S. W., Leevy, C. M. and Davidson, C. S., “Indocyanine green: observations on its physical properties, plasma decay, and hepatic extraction,” J Clin Invest 39, 592-600 (1960).
Desmettre, T., Devoisselle, J. M. and Mordon, S., “Fluorescence properties and metabolic features of indocyanine green (ICG) as related to angiography,” Sury Ophthalmol 45, 15-27 (2000).
Uesaka, K., Nimura, Y. and Nagino, M., “Changes in hepatic lobar function after right portal vein embolization. An appraisal by biliary indocyanine green excretion,” Ann Surg 223, 77-83 (1996).
Ishizawa, T., Bandai, Y. and Kokudo, N., “Fluorescent cholangiography using indocyanine green for laparoscopic cholecystectomy: an initial experience,” Arch Surg 144, 381-382 (2009).
Ishizawa, T., Tamura, S., Masuda, K., Aoki, T., Hasegawa, K., Imamura, H., Beck, Y. and Kokudo, N., “Intraoperative fluorescent cholangiography using indocyanine green: a biliary road map for safe surgery,” J Am Coll Surg 208, e1-4 (2009).
Sou, S., Matsui, T., Yao, T., Naito, M., Yorioka, M., Beppu, T., Nagahama, T. and Futami, K., “Differentiating enterocutaneous fistulae from suture abscesses complicating Crohn's disease using oral administration of indocyanine green,” J Gastroenterol Hepatol 21, 1850-1853 (2006).
Yaseen, M. A., Yu, J., Wong, M. S. and Anvari, B., “In-vivo fluorescence imaging of mammalian organs using charge-assembled mesocapsule constructs containing indocyanine green,” Opt Express 16, 20577-20587 (2008).
Lee, A. W., Ram, M. D., Shih, W. J. and Murphy, K., “Technetium-99m BIDA biliary scintigraphy in the evaluation of the jaundiced patient,” J Nucl Med 27, 1407-1412 (1986).
Krishnamurthy, G. T. and Turner, F. E., “Pharmacokinetics and clinical application of technetium 99m-labeled hepatobiliary agents,” Semin Nucl Med 20, 130-149 (1990).
Tennvassas, T. L. and Skretting, A., “Methods for automatic detection of peristaltic waves in the small intestine,” Eur J Nucl Med 25, 1531-1536 (1998).
Krishnamurthy, G. T. and Krishnamurthy, S., “Extended application of 99 mTc-mebrofenin cholescintigraphy with cholecystokinin in the evaluation of abdominal pain of hepatobiliary and gastrointestinal origin,” Nucl Med Commun 31, 346-354.
Torjman, M. C., Joseph, J. I., Munsick, C., Morishita, M. and Grunwald, Z., “Effects of isoflurane on gastrointestinal motility after brief exposure in rats,” Int J Pharm 294, 65-71 (2005).
Marshall, M. V., Rasmussen, J. C., Tan, I. C., Aldrich, M. B., Adams, K. E., Wang, X., Fife, C. E., Maus, E. A., Smith, L. A. and Sevick-Muraca, E. M., “Near-infrared fluorescence imaging in humans with indocyanine green: a review and update,” the Open Surgical Oncology Journal 2, 12-25 (2010).
Sevick-Muraca, E. M., Sharma, R., Rasmussen, J. C., Marshall, M. V., Wendt, J. A., Pham, H. Q., Bonefas, E., Houston, J. P., Sampath, L., Adams, K. E., Blanchard, D. K., Fisher, R. E., Chiang, S. B., Elledge, R. and Mawad, M. E., “Imaging of lymph flow in breast cancer patients after microdose administration of a near-infrared fluorophore: feasibility study,” Radiology 246, 734-741 (2008).
Rasmussen, J. C., Tan, I. C., Marshall, M. V., Adams, K. E., Kwon, S., Fife, C. E., Maus, E. A., Smith, L. A., Covington, K. R. and Sevick-Muraca, E. M., “Human Lymphatic Architecture and Dynamic Transport Imaged Using Near-infrared Fluorescence,” Transl Oncol 3, 362-372 (2010).
Maus, E. A., Tan, I. C., Rasmussen, J. C., Marshall, M. V., Fife, C. E., Smith, L. A., Guilliod, R. and Sevick-Muraca, E. M., “Near-infrared fluorescence imaging of lymphatics in head and neck lymphedema,” Head Neck (2010).
Tan, I. C., Maus, E. A., Rasmussen, J. C., Marshall, M. V., Adams, K. E., Fife, C. E., Smith, L. A. and Sevick-Muraca, E. M., “Near-infrared fluorescence assessment of contractile function following manual lymphatic drainage,” APMR Accepted for publication, (2010).
Yaseen, M. A., Yu, J., Wong, M. S. and Anvari, B., “In-vivo fluorescence imaging of mammalian organs using charge-assembled mesocapsule constructs containing indocyanine green,” Opt Express 16, 20577-20587 (2008).
Marshall, M. V., Rasmussen, J. C., Tan, I. C., Aldrich, M. B., Adams, K. E., Wang, X., Fife, C. E., Maus, E. A., Smith, L. A. and Sevick-Muraca, E. M., “Near-infrared fluorescence imaging in humans with indocyanine green: a review and update,” the Open Surgical Oncology Journal 2, 12-25 (2010).
Zhu, B., Rasmussen, J. C., Lu, Y. and Sevick-Muraca, E. M., “Reduction of excitation light leakage to improve near-infrared fluorescence imaging for tissue surface and deep tissue imaging,” Med Phys 37, 5961-5970.
Der-Silaphet, T., Malysz, J., Hagel, S., Larry Arsenault, A. and Huizing a, J. D., “Interstitial cells of cajal direct normal propulsive contractile activity in the mouse small intestine,” Gastroenterology 114, 724-736 (1998).
Der, T., Bercik, P., Donnelly, G., Jackson, T., Berezin, I., Collins, S. M. and Huizing a, J. D., “Interstitial cells of cajal and inflammation-induced motor dysfunction in the mouse small intestine,” Gastroenterology 119, 1590-1599 (2000).
Bercik, P., Wang, L., Verdu, E. F., Mao, Y. K., Blennerhassett, P., Khan, W. I., Kean, I., Tougas, G. and Collins, S. M., “Visceral hyperalgesia and intestinal dysmotility in a mouse model of postinfective gut dysfunction,” Gastroenterology 127, 179-187 (2004).
Ailiani, A. C., Neuberger, T., Brasseur, J. G., Banco, G., Wang, Y., Smith, N. B. and Webb, A. G., “Quantitative analysis of peristaltic and segmental motion in vivo in the rat small intestine using dynamic MRI,” Magn Reson Med 62, 116-126 (2009).
LI-COR Biosciences Application Note, “In Vivo Animal Imaging Diet Considerations”, 2009 Kwon, S. and Sevick-Muraca, E. M., “Non-invasive imaging of murine intestinal motility” Neurogastroenterol Motil. 23 (9):881-e344, 2011.

1. A method of non-invasively imaging gastrointestinal motility in a subject having an abdominal surface and a gastrointestinal tract, the method comprising:
a) selecting a subject that has been administered an animal foodstuff comprising at least one auto fluorescent compound;
b) illuminating the abdominal surface with an excitation light to excite the auto fluorescent compound;
c) continuously, non-invasively detecting fluorescent emissions from the auto fluorescent compound to image at least a region of the gastrointestinal tract of the subject;
d) capturing a plurality of images of said one or more regions of the gastrointestinal tract of the subject, based on said detected auto fluorescent emissions; and
e) tracking in vivo propulsion of said auto fluorescent compound through the one or more regions of the gastrointestinal tract, based on said captured images.

2. The method of claim 1, wherein (a) selecting a subject comprises:
i) administering an animal foodstuff comprising at least one auto fluorescent compound to the subject; and
ii) allowing sufficient time for said foodstuff to enter into the gastrointestinal tract of the subject;

3. The method of claim 1, wherein the auto fluorescent compound has an excitation wavelength ranging from about 700 nm to about 900 nm.

4. The method of claim 1, wherein (c) comprises using an intensified charge-coupled camera.

5. The method of claim 1, wherein (b) comprises illuminating the tissue surface with an excitation light source selected from group consisting of laser diodes, semiconductor laser diodes, gas lasers, light emitting diodes, and combinations thereof.

6. A method of non-invasively assessing gastrointestinal motility in a subject, utilizing the method of claim 1.

7. A method of non-invasively measuring propulsion of the gastrointestinal system of a subject comprising measuring at least one of contraction frequency and the propagation velocity, utilizing the method of claim 1.

8. A method of non-invasively identifying a subject suffering from, at risk of or in need of therapy for a gastrointestinal motility disorder, utilizing the method of claim 1.

9. The method of claim 8, further comprising identifying a subject in need of surgery for a gastrointestinal blockage.

10. A method of accelerating both preclinical and clinical drug or therapy discovery, validation and approval by providing a convenient method for longitudinally assessing gastrointestinal motility in veterinary or human subjects, utilizing the method of claim 1.

11. A method of non-invasively identifying the portion of a subject's intestinal tract at risk of developing a gastrointestinal motility disorder or in need of therapy due to a gastrointestinal motility disorder, using the method of claim 1.

12. The method of claim 11, further comprising identifying the particular portion of a subject's intestinal tract in need of surgery due to a gastrointestinal motility disorder.

13. A method of non-invasively determining the efficacy of a therapy for a gastrointestinal motility disorder in a subject being treated for a gastrointestinal motility disorder, using the method of claim 1.

14. A method of non-invasively monitoring the progress of a subject being treated for a gastrointestinal motility, using the method of claim 1.

15. The method of claim 1 wherein, in d), capturing said plurality of images comprises a camera integration time in the range of about 10 milliseconds to about 1 second or about 100 milliseconds to about 800 milliseconds.

16. The method of claim 15, wherein capturing said plurality of images comprises a camera integration time greater than 200 milliseconds.

17. The method of claim 1, comprising (f) calculating intestinal flow velocity, intestinal segmental contraction rate or intestinal peristaltic wave rate to assess intestinal functionality.

18. A method of non-invasively assessing gastrointestinal function in a subject, comprising:
a) selecting a subject that has been administered an imaging packet comprising at least one auto fluorescent compound having a characteristic excitation wavelength and a characteristic fluorescence emission wavelength;
b) noninvasively illuminating a tissue surface of the intestinal tract on the subject's body with radiation at said characteristic excitation wavelength;
c) noninvasively detecting fluorescence emissions from said compound within a region of interest and capturing a plurality of fluorescence images for an interval of time;
d) using said fluorescence images to visualize gastrointestinal motion in a region of interest and to track the location of said compound in said region of interest as a function of time to obtain a set of tracked image locations as a function of time;
e) determining from said tracked locations as a function of time an initial intestinal propulsion measurement;
f) optionally, comparing said initial intestinal propulsion measurement to a subsequently determined intestinal propulsion measurement; and
g) determining from the results of d) and e), and f), if present, the functionality of a gastrointestinal tract in said region of interest in said subject.

19-35. (canceled)

36. A method of non-invasively imaging gastrointestinal motility in a subject, the method comprising:
a) selecting a subject that has been administered at least one packet comprising a fluorescent agent that when administered in vivo is excreted in bile;
b) illuminating the abdominal tissue surface with an excitation light to excite the fluorescent agent;
c) continuously, non-invasively detecting fluorescent emissions from the fluorescent agent to image at least a region of the intestinal tract;
d) capturing a plurality of images of said one or more regions of the intestinal tract, based on said detected fluorescent images; and
e) tracking in vivo propulsion of said at least one packet through the one or more regions of the intestinal tract, based on said captured images.

37-55. (canceled)

56. A method of non-invasively assessing gastrointestinal function in a subject, comprising:
a) selecting a subject that has been administered at least one packet comprising a fluorescent agent having a characteristic excitation wavelength and a characteristic fluorescence emission wavelength and that when administered in vivo is excreted in bile;
b) noninvasively illuminating a tissue surface of the intestinal tract on the subject's body with radiation at said characteristic excitation wavelength;
c) noninvasively detecting fluorescence emissions from said agent within a region of interest and capturing a plurality of fluorescence images for an interval of time;
d) using said fluorescence images to visualize gastrointestinal motion in a region of interest and to track the location of said agent in said region of interest as a function of time to obtain a set of tracked image locations as a function of time;
e) determining from said tracked locations as a function of time an initial intestinal propulsion measurement;
f) optionally, comparing said initial intestinal propulsion measurement to a subsequently determined intestinal propulsion measurement; and
g) determining from the results of d) and e), and f) if present, the functionality of a gastrointestinal tract in said region of interest in said subject.

57-75. (canceled)
